(19) United States
(12) Patent Application Publication
(10) Pub. No.: US 2020/0054501 A1
(43) Pub. Date: Feb. 20, 2020

(54) NONWOVEN FABRIC

(71) Applicant: Kao Corporation, Tokyo (JP)

(72) Inventors: Yoshihiko SETO, Utsunomiya-shi, Tochigi (JP); Yoshihiko KINUGASA, Utsunomiya-shi, Tochigi (JP); Masahiro TANIGUCHI, Utsunomiya-shi, Tochigi (JP)

(73) Assignee: Kao Corporation, Tokyo (JP)

(21) Appl. No.: 16/609,300

(22) PCT Filed: Jul. 17, 2018
(86) PCT No.: PCT/JP2018/026731

§ 371 Date: Oct. 29, 2019

(30) Foreign Application Priority Data
Aug. 31, 2017 (JP) 2017-168003

(51) Int. Cl. A61F 13/511; D04H 1/54

(52) U.S. Cl. CPC A61F 13/511; D04H 1/74; D04H 1/54

(57) ABSTRACT

A nonwoven fabric 10, comprising a movable layer 4 having front and back surfaces 10SA and 10SB, wherein the movable layer 4 has a movable zone, in which one surface of the front and back surfaces is movable by 5 mm or more in a direction along the one surface, relative to the other surface.

### FIELD OF THE INVENTION

The present invention relates to a nonwoven fabric.

### BACKGROUND OF THE INVENTION

A nonwoven fabric is often used in a diaper for a baby, a diaper for an adult, a sanitary item, an eye mask, a mask or the like. Technology on providing this nonwoven fabric with various functions is known.

For example, in the nonwoven fabric described in Patent Literature 1, a plurality of first projecting portions projecting on one surface side, and second projecting portions projecting on a surface side opposite to the one surface are alternately extend and continue in two different directions in a plane through a wall portion having an annular structure. In this nonwoven fabric, in order to realize a satisfactory soft skin-touch texture of the nonwoven fabric by point skin contact, fiber density in the first projecting portion is set to be lower than fiber density in the second projecting portion.

In order to obtain bulkiness and strechability, the fabric described in Patent Literature 2 is formed of a nonwoven fabric having at least a first fiber and a second fiber, whose difference in shrinkage ratio between the first and second fibers is set to at least about 8%.

In the nonwoven fabric described in Patent Literature 3, convex line portions are enabled to easily follow movement of wearer's skin by forming fiber density of a side portion zone present between a top portion zone constituting a top portion of the convex line portion and a bottom portion zone constituting a bottom portion of a concave line portion smaller than fiber density in the top portion zone and fiber density in the bottom portion zone.

### CITATION LIST

### Patent Literatures

Patent Literature 1: JP-A-2012-136791 (“JP-A” means unexamined published Japanese patent application)

Patent Literature 2: JP-A-2009-510278

Patent Literature 3: JP-A-2016-079529

### SUMMARY OF THE INVENTION

The present invention provides a nonwoven fabric, comprising a movable layer having front and back surfaces of the nonwoven fabric, wherein the movable layer has a movable zone, in which one surface of the front and back surfaces is movable by 5 mm or more in a direction along the one surface, relative to the other surface.

Other and further objects, features and advantages of the invention will appear more fully from the following description, appropriately referring to the accompanying drawings.

### BRIEF DESCRIPTION OF DRAWINGS

FIG. 1 is a view of a partial cross section schematically showing a state of wearing a diaper using one preferable embodiment of the nonwoven fabric according to the present invention as a topsheet.

FIG. 2 is a schematic configuration view showing one example of a method for measuring a moving range of a nonwoven fabric in a direction along a front surface thereof, in which FIG. 2(A) is a view showing a state before measurement, and FIG. 2(B) is a view showing a measuring state.

FIG. 3 is a perspective view of a partial cross section showing a specific example of one preferable embodiment of the nonwoven fabric according to the present invention.

FIG. 4 is a cross-sectional view taken along an F1-F1 line of the nonwoven fabric shown in FIG. 3.

FIG. 5 is a cross-sectional view taken along an F2-F2 line of the nonwoven fabric shown in FIG. 3.

FIG. 6 is a view showing a method for measuring the number of fused points between constituent fibers, in which FIG. 6(A) is a drawing substitute photograph showing a bird's eye view of a nonwoven fabric, and FIG. 6(B) is a plane view schematically showing an image of the P portion in FIG. 6(A) by a scanning electron microscope.

FIG. 7 is a view showing a method for measuring the number of constituent fibers, which is a plane view schematically showing an image of the P portion in FIG. 6(A) by a scanning electron microscope.

FIG. 8 is a view showing a method for measuring the fiber orientation parameter, which is a plane view schematically showing an image of the P portion in FIG. 6(A) by a scanning electron microscope.

FIG. 9 is a view of a partial cross section schematically showing another preferable embodiment of the nonwoven fabric according to the present invention.

FIG. 10 is a graph showing recoverability after one day compression of a nonwoven fabric, in which sheath-core type conjugate fibers having core resin component of is polyethylene terephthalate and sheath resin component of polyethylene are used.

FIG. 11 is a perspective view with partial cutaway schematically showing a specific example of a diaper when the nonwoven fabric according to the present invention is used as a topsheet.

FIG. 12 is an explanatory view schematically showing one preferable example of a method for producing a nonwoven fabric in the embodiment, in which FIG. 12(A) is an explanatory view showing a step of arranging a fiber web on a support male material and pushing a support female material into the support male material from above the fiber web, FIG. 12(B) is an explanatory view showing a step of blowing first hot air from above a support female material and shaping the fiber web, and FIG. 12(C) is an explanatory view showing a step of removing the support female material, and blowing second hot air from above a shaped fiber web to fuse fibers to each other.

### DESCRIPTION OF EMBODIMENTS

The present invention relates to a nonwoven fabric having excellent followability to a skin surface.

Friction arising between a front or back surface of a nonwoven fabric and a wearer's skin surface upon movement of the skin surface may lead to chafing. With a view to protecting the skin surface, a nonwoven fabric is desired that, by more flexibly deforming to better follow the skin surface, can still more effectively avoid chafing.

Regarding this point, the nonwoven fabric described in Patent Literature 1, for example, can suppress pressure-induced shape deformation of the nonwoven fabric as a whole to a low level, but there is room for improvement in the followability of the nonwoven fabric in a direction along its surface relative to movement of the skin surface. Further, the nonwoven fabric described in Patent Literature 2 is a nonwoven fabric having a flat surface without concavo-convex shape on either surface, so that the followability to a wearer's undulating skin surface is low and friction arising between the nonwoven fabric surface and the skin surface is known to increase and induce the aforesaid chafing. Furthermore, in the nonwoven fabric described in Patent Literature 3, convex line portions are formed to easily follow the movement of wearer's skin, but there is room for further improvement in the followability of the nonwoven fabric to the movement of the skin surface.

A nonwoven fabric according to the present invention has excellent followability to a skin surface.

A preferred embodiment of the nonwoven fabric according to the present invention will be explained below, referring to figures. However, the present invention should not be intended to be limited thereto.

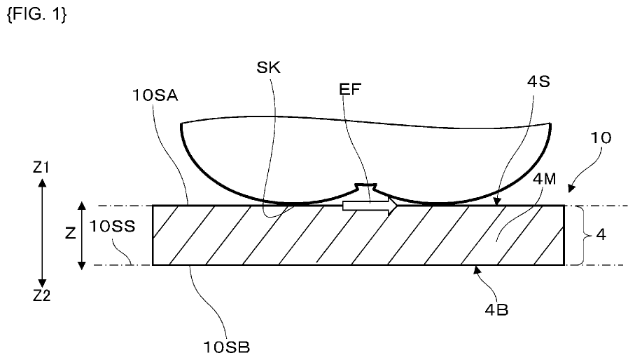

As shown in FIG. 1, a nonwoven fabric 10 of the embodiment comprises front and back surfaces. The embodiment will be described by taking front and back surfaces as a front surface 10SA and a back surface 10SB for a surface on a side opposite to the front surface 10SA. Further, thickness direction of the nonwoven fabric 10 is taken as a Z direction. In the embodiment, unless otherwise specified, the front surface 10SA is shown as a surface to be visually observed (an observation surface), but the nonwoven fabric according to the present invention is not limited thereto, and the back surface 10SB may be taken as the surface to be visually observed (the observation surface).

The nonwoven fabric 10 comprises a movable layer 4 having a front surface 10SA and a back surface 10SB. Specifically, the movable layer 4 comprises the regions on a front surface side 4S, a back surface side 4B and an internal side 4M of the movable layer in thickness direction of the nonwoven fabric 10. The region on the front surface side 4S means a region in which visible fibers exist in thickness direction when viewed from the front surface 10SA of the nonwoven fabric 10, and the region on the back surface side 4B means a region in which the visible fibers exist in thickness direction when viewed from the back surface 10SB of the nonwoven fabric 10. The region on the internal side 4M of the movable layer means a region interposed between the front surface side 4S and the back surface side 4B in thickness direction. That is, the region on the front surface side 4S of the movable layer 4 includes the front surface 10SA of the nonwoven fabric 10, and the region on the back surface side 4B of the movable layer 4 includes the back surface 10SB thereof.

The movable layer 4 has a movable zone where plane direction movement of 5 mm or more is possible between one surface and the other surface of the nonwoven fabric, namely between the front surface 10SA and the back surface 10B and vice versa (size of the movable zone hereinafter also being called “moving range” or “movable amount”.). The movable amount of the movable layer 4 is preferably 6 mm or more, and more preferably 7 mm or more. Upper limit of the movable amount is not particularly defined, but from a viewpoint of preventing sticking onto the skin, the upper limit is 10 mm or less, preferably 9 mm or less, and more preferably 8 mm or less.

In the movable zone in the movable layer 4, the front surface 10SA and the back surface 10SB of the nonwoven fabric 10 may move in directions opposite to each other. Such movement is enabled by a configuration in which the internal side 4M of the movable layer 4 is formed into an intermediate region which has high deformability that allows the internal side 4M to start moving by a force equal to or less than friction force between the skin and the nonwoven fabric 10.

Although the movable layer 4 is described in the following regarding a case where the front surface 10SA is movable relative to the back surface 10SB in a direction along the front surface 10SA, the same description also applies to a case where the back surface 10SB is movable relative to the front surface 10SA.

FIG. 1 shows a movable layer 4 in which the front surface 10SA of the nonwoven fabric 10 is in contact with a skin surface SK, and movable in a direction along the front surface 10SA relative to the back surface 10SB. The direction along the front surface 10SA means a direction along a virtual plane disposed so as to be brought into contact with the front surface 10SA of the nonwoven fabric 10 when the nonwoven fabric 10 is extended and the back surface 10SB side thereof is placed on a plane. The direction therealong means a direction in parallel thereto. The above-described movable layer 4 means a layer in which, when external force EF (shown by an arrow EF in FIG. 1) is applied thereto in the direction along the front surface 10SA of the nonwoven fabric 10, the front surface 10SA moves relative to the back surface 10SB in the direction in which the external force EF is applied thereto. The nonwoven fabric 10 as a whole preferably serves as the movable layer 4.

Specific examples of a preferable embodiment of the movable layer 4 include a configuration having a concavo-convex portion and a wall portion as described later. When the movable layer 4 has a convex portion on the front surface 10SA or the back surface 10SB of the nonwoven fabric 10, if a moving range of the front surface is taken as D, an apparent thickness is taken as t, and an external angle is taken as e, each parameter has a relationship represented by the following formula (1).

D=|t·cos θ| (1)

In addition, even when the nonwoven fabric 10 does not have concavo-convex shape and both the front surface 10SA and the back surface 10SB have a flat surface, the nonwoven fabric 10 can have the movable layer 4. In this case, the moving range of the front surface 10SA is not limited by the apparent thickness of the nonwoven fabric 10. Even if fibers in the movable layer 4 are folded and the apparent thickness is reduced, the moving range may be secured. That is, the front surface may be movable in a level equal to or more than the apparent thickness. The apparent thickness is thickness of the nonwoven fabric 10 measured by the measuring method described below.

Movability of the movable layer 4 is attributable to the fibers on the internal side 4M of the movable layer being free to move. For example, the movability is due to presence of a region in which the number of fused points between constituent fibers per unit area is lower on the internal side 4M of the movable layer than the number thereof on the front surface side 4S and the back surface side 4B of the movable layer, presence of a region in which the number of constituent fibers per unit area is lower therein, presence of a region in which the fibers are oriented in a vertical direction therein, or the like. Thus, in following movement of the skin surface SK, the front surface 10SA moves relative to the skin surface SK without sliding. Furthermore, the front surface 10SA starts moving under a force smaller than the friction force working between the front surface 10SA and the skin surface SK. Therefore, even if no special measure is taken with respect to the front surface 10SA of the nonwoven 10 to enhance friction force between the front surface 10SA and the skin surface SK, the front surface 10SA nevertheless follows the skin surface SK owing to the movability of the movable layer 4. The aforesaid movability of the movable layer 4 enables the front surface 10SA of the nonwoven fabric 10 also to follow random movement of the skin surface SK. Abrasion of the skin surface SK caused by the front surface 10SA of the nonwoven fabric 10 can be minimized by such followability of the nonwoven fabric 10. Moreover, even if the movable layer 4 of the nonwoven fabric 10 is once bent and stays bent, the followability is still ensured by the movability of the movable layer 4.

### [Method for Measuring Moving Range of Front Surface 10SA of Nonwoven Fabric 10]

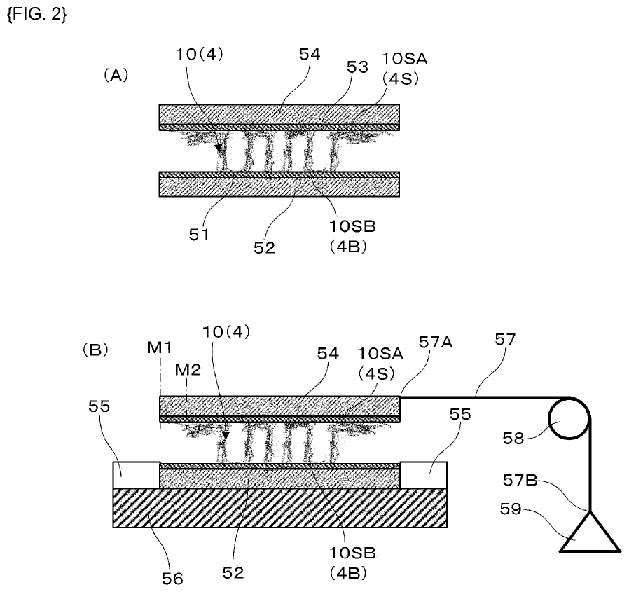

As shown in FIG. 2, measurement is carried out as follows.

(i) Preparation of measuring sample:

As a measuring sample, a nonwoven fabric sample having a size of 50 mm×50 mm is arranged. As shown in FIG. 2(A), an adhesive is applied onto a whole surface of a mounting paper, which is called as a mount 52 on a back surface side of the nonwoven fabric sample, to form an adhesive layer 51, and the back surface 10SB of the nonwoven fabric sample is adhered onto the adhesive layer 51 and fixed thereonto. Bond G103 manufactured by Konishi Co., Ltd. is used as the adhesive, and 0.5 g is applied thereonto. Further, an adhesive identical to the above-described adhesive is applied onto a whole surface of another mounting paper, which is called as a mount 54 on a front surface side of the nonwoven fabric sample, to form an adhesive layer 53, and the front surface 10SA of the nonwoven fabric sample is adhered onto the adhesive layer 53 and fixed thereonto. Furthermore, when the nonwoven fabric is unable to be collected at the size of 50 mm×50 mm, a plurality of fabrics are aligned so as to have the above-described size, and to be adhered onto the mount.

In addition, when a nonwoven fabric assembled into a commercially available absorbent article is applied as a measuring object, the nonwoven fabric is carefully peeled and removed from the absorbent article by using a cold spray to prepare the above-described measuring sample. At this time, when a hot-melt adhesive is attached to the sample, the hot-melt adhesive is removed by using an organic solvent. This means is the same as all on the sample used in other measurements in nonwoven fabrics in the description.

(ii) Measurement of moving range:

Next, as shown in FIG. 2(B), the mount 52 on the back surface side is fixed on a base 56 for measurement by using a fixing device 55. One end 57A of a string 57 for applying tensile force to the front surface 10SA of the nonwoven fabric sample in one direction along the front surface 10SA is attached to the mount 54 on the front surface side. The other end 57B of the string 57 is vertically dangled downward through a rotatable pulley 58. During measurement, a weight 59 is attached to the other end 57B of the string 57 so as to be suspended. Accordingly, when the weight 59 is attached to the other end 57B of the string 57, the string 57 pulls the mount 54 on the front surface side in a direction along the front surface of the nonwoven fabric sample (a right direction from a viewer in FIG. 2(B)) by gravity of the weight 59.

With regard to the measurement, first, an initial position of the nonwoven fabric sample is measured in a state without attaching the weight 59 to obtain a measured value M1. Then, the weight 59 (50 g) is attached thereto, and the weight 59 is gently released. Thus, the front surface 10SA of the nonwoven fabric 10 is pulled in the direction along the front surface 10SA (pulley direction) by the weight 59. FIG. 2(B) shows a state immediately before pulling the front surface 10SA. Upon pulling the front surface 10SA, shear stress (200 Pa under the above-described conditions) is applied to the front surface 10SA of the nonwoven fabric sample.

The weight 59 is released and the movement of the front surface 10SA of the nonwoven fabric sample is stopped, and then a stopping position of the nonwoven fabric sample is measured to obtain a measured value M2. Then, a moved amount of the front surface 10SA of the nonwoven fabric sample is obtained by calculating a difference between the measured value M2 and the measured value M1, and this moved amount is taken as the moving range of the front surface 10SA of the nonwoven fabric 10.

Next, a preferable embodiment of the nonwoven fabric 10 is explained.

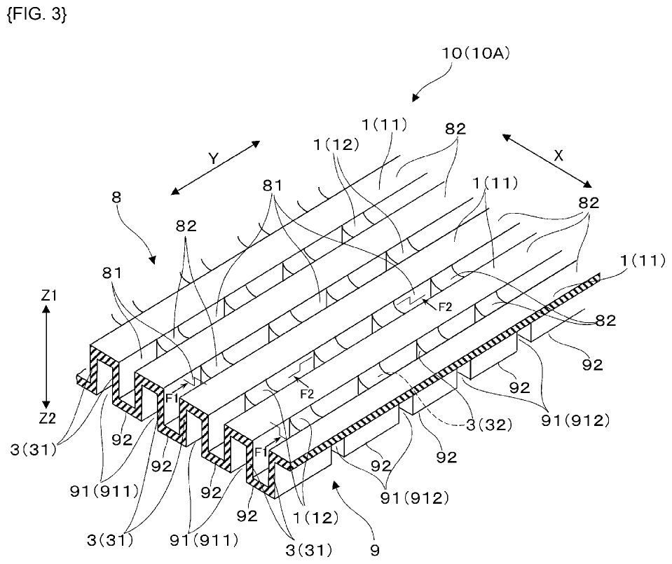

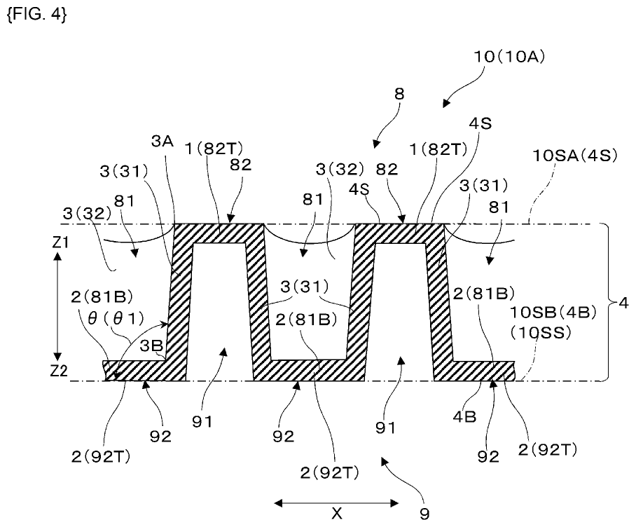

FIGS. 3 to 5 show a preferable embodiment of the nonwoven fabric 10 (nonwoven fabric 10A). The nonwoven fabric 10A comprises a concavo-convex portion 8 on a first surface side Z1, and comprises a concavo-convex portion 9 on a second surface side Z2. The concavo-convex portion 8 has a concave portion 81 and a convex portion 82 when viewed from the first surface side Z1 side. Here, the embodiment will be described by taking the back surface 10SB in the above-mentioned method for measuring the moving range of the front surface 10SA of the nonwoven fabric 10 as the second surface side Z2, and the front surface 10SA as the first surface side Z1. A plane when the nonwoven fabric is extended and placed on the plane is taken as a “reference surface”. In this case, a surface on the second surface side Z2 when the nonwoven fabric 10 is extended and placed on the plane with the second surface side Z2 of the nonwoven fabric 10 downward is taken as a nonwoven fabric reference surface 10SS (hereinafter, also referred to as a reference surface 10SS) (see FIG. 4). Accordingly, the back surface 10SB and the reference surface 10SS become the same plane (see FIG. 4). That is, the convex portion 82 is projected from the reference surface 10SS in thickness direction of the nonwoven fabric 10 in a ridge form. Moreover, the concavo-convex portion 9 has a concave portion 91 and a convex portion 92 when viewed from the second surface side Z2 side. Here, the concave portion 81 and the convex portion 92 are in a front-back relation, and the concave portion 91 and the convex portion 82 are in the front-back relation. In addition, the back surface 10SB side in the measuring method may be taken as the first surface side Z1, and in this case, the concavo-convex portion 8 serves as the concavo-convex portion 9, and the concave portion 81 serves as the convex portion 92.

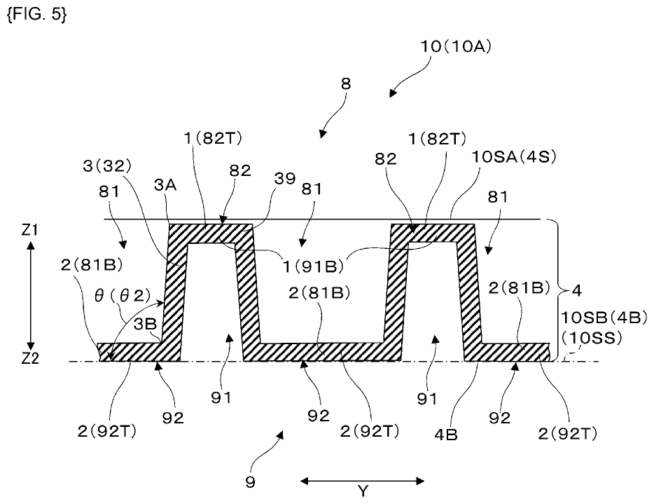

As shown in FIGS. 4 and 5, the concavo-convex portion 8 and the concavo-convex portion 9 comprise the following configuration.

The concavo-convex portion 8 has a bottom portion 81B (hereinafter, also referred to as a concave bottom portion 81B) of the concave portion 81, a top portion 82T (hereinafter, also referred to as a convex top portion 82T) of the convex portion 82, and a wall portion 3 linking the convex top portion 82T and the concave bottom portion 81B. The concave bottom portion 81B is formed of an outer surface fiber layer 2 forming the second surface side Z2. The convex top portion 82T is formed of an outer surface fiber layer 1 forming the flat surface on the first surface side Z1. The wall portions 3 form side surface portions of the concave portion 81 and the convex portion 82, and are a common wall dividing the concave portion 81 and the convex portion 82.

Moreover, the concavo-convex portion 9 has a bottom portion 91B (hereinafter, also referred to as a concave bottom portion 91B) of the concave portion 91, a top portion 92T (hereinafter, also referred to as a convex top portion 92T) of the convex portion 92, and a wall portion 3 linking the convex top portion 92T and the concave bottom portion 91B. The concave bottom portion 91B is formed of an outer surface fiber layer 1 on the first surface side Z1. The convex top portion 92T is formed of an outer surface fiber layer 2 forming the flat surface on the second surface side Z2. The wall portions 3 form side surface portions of the concave portion 91 and the convex portion 92, and are a common wall dividing the concave portion 91 and the convex portion 92.

In addition thereto, the top portion 82T and the bottom portion 91B are formed of the outer surface fiber layer 1 common thereto. The top portion 92T and the bottom portion 81B are formed of the outer surface fiber layer 2 common thereto.

Moreover, the concave portion 91 has a concave portion 911 in which a first outer surface fiber layer 11 serves as a bottom portion, and a concave portion 912 in which an outer surface fiber layer 12 serves as the bottom portion, in corresponding to the first outer surface fiber layer 11 and the second outer surface fiber layer 12 of the outer surface fiber layer 1, respectively. Then, on the second surface side Z2, the concave portion 911 is communicated in a Y direction, the concave portion 912 is communicated in an X direction, and the concave portion 911 is communicated with the concave portion 912.

Further, the wall portions 3 form an outer wall surrounding four directions of the concave portion 81 on the first surface side Z1. That is, an inside of the concave portion 81 surrounded by the wall portions 3 forms an independent space. In the embodiment, a box-shaped space is formed by four wall portions 3. However, the number of the wall portions 3 surrounding the concave portion 81 and a concave portion shape formed by the wall portions 3 are not limited thereto.

Further, when the second surface side Z2 is taken as the reference surface 10SS, an external angle θ of the wall portion 3 of the convex portion 82 is preferably 110° or less.

The external angle θ of the wall portion 3 forming the convex portion 82 is defined as an angle outside the convex portion 82, in which the angle is formed between the reference surface 10SS and a straight line passing through an uppermost end portion and a lowermost end portion of the wall portion 3 in a vertical cross section in a center of the concave portion 81 of the concavo-convex portion 8 along one direction of the nonwoven fabric 10.

The external angle θ of the wall portion 3 forming the convex portion 82 shown in FIG. 3 has an external angle θ1 (FIG. 4) between the reference surface 10SS and a straight line passing through an upper end portion and a lower end portion of the wall portion 3 in a vertical cross section in the center of the concave portion 81 of the concavo-convex portion 8 along one direction of the nonwoven fabric 10, and an external angle θ2 (FIG. 5) between the reference surface 10SS and a straight line passing through an upper end portion and a lower end portion of the wall portion 3 in a vertical cross section in the center of the concave portion 81 of the concavo-convex portion 8 along a direction perpendicular to the one direction of the nonwoven fabric 10. The external angles θ1 and θ2 each are the external angle measured from a direction perpendicular to each other with regard to the vertical cross section in the X direction along an F1-F1 line and the vertical cross section in the Y direction along an F2-F2 line in FIG. 3. Both the external angles θ1 and θ2 are preferably within the following specified value. In addition, when the first surface side Z1 is taken as the reference surface 10SS, the external angle θ of the wall portion 3 of the convex portion 92 is preferably 110 degrees or less.

From a viewpoint of forming the movable layer 4 into a material having the above-mentioned movable zone, the external angle θ is preferably 110° or less, more preferably 100° or less, and further preferably 90° or less. Moreover, the external angle θ is preferably 60° or more, more preferably 70° or more, and further preferably 80° or more. The external angle θ is set to the above-mentioned upper limit or less, so that the wall portion 3 as a whole becomes easily movable in an inclined manner from a starting point of the nonwoven fabric reference surface 10SS by external force applied to the front surface 10SA (front surface of the outer surface fiber layer 1) in a direction along the front surface, thereby increasing the moving amount of the front surface 10SA, and a sufficient movable range is obtained. On the other hand, the convex portions 82 are separated from each other by setting the external angle θ to the above-mentioned lower limit or more, and a concavo-convex structure in a plane view is obtained.

In addition, between an upper end portion 3A and a lower end portion 3B of the wall portion 3, even if the external angle θ of the wall portion 3 relative to the nonwoven fabric reference surface 10SS is partially outside the above-described range, such a case is allowed. For example, between the upper end portion 3A and the lower end portion 3B of the wall portion 3, the wall portion 3 may have a wavy shape when viewed in the above-described vertical cross section.

The wall portions 3 surrounding the concave portion 81 from side portions are preferably inclined in a comparable level, respectively. More specifically, values of the external angles θ of the respective wall portions are preferably the same.

For example, the external angle θ (for example, θ1) measured from one direction of the wall portion is preferably in the comparable level relative to the external angle θ (for example, θ2) measured from a direction perpendicular to the one direction.

By “comparable level” is meant that a difference between both the external angles θ1 and θ2 is 0° or more and 10° or less, preferably 8° or less, more preferably 6° or less, and further preferably 4° or less.

### [Method for Measuring External Angle θ]

A measuring sample is prepared by the method shown in (i) Preparation of measuring sample in [Method for measuring moving range of front surface 10SA of nonwoven fabric 10] mentioned above.

Next, the measuring sample of the nonwoven fabric 10 is cut from a surface on the first surface side Z1 toward a surface on the second surface side Z2 or from the surface on the second surface side Z2 toward the surface on the first surface side Z1 so as to include the concavo-convex portion 8 or the concavo-convex portion 9 to obtain the vertical cross section (F1-F1 cross section (see FIG. 4) or F2-F2 cross section (see FIG. 5)). At this time, each cross section is formed so as to include the concave portion 81, the convex portion 82 and the wall portion 3, or the concave portion 91, the convex portion 92 and the wall portion 3. Next, the cut sample is placed in such a manner that the reference surface 10SS of the nonwoven fabric 10 becomes horizontal, and each vertical cross section described above is photographed so as to include the concave portion 81, the convex portion 82 and the wall portion 3 or the concave portion 91, the convex portion 92 and the wall portion 3 to obtain a cross-sectional image. The external angle θ of the wall portion 3 is measured from each cross-sectional image photographed. As one method for measuring the external angle θ, a straight line passing through the upper end portion 3A and the lower end portion 3B of the wall portion 3, and a reference line representing the reference surface 10SS are drawn on the cross-sectional image, and the external angle between the straight line and the reference line is measured, for example, by a protractor to obtain the external angle θ of the wall portion 3. Also when a visually observed surface of the wall portion 3 has a concavo-convex surface, and is not flat, the external angle can be measured in the same manner described above.

In the nonwoven fabric 10, the number of fused points (fused point number) between the constituent fibers per unit area in the region on the internal side 4M of the movable layer 4 (see FIG. 1) is preferably lower than the number of fused points between the constituent fibers per unit area in either or both of the regions on the front surface side 4S and on the back surface side 4B of the movable layer 4, respectively.

The internal side 4M of the movable layer becomes further easily movable in the direction along the surface than the front surface side 4S or the back surface side 4B by having the above-described relationship. The reason is that the internal side 4M of the movable layer is less inhibited in the movement of the constituent fibers by the fused points of the constituent fibers, and becomes easily movable. Thus, the front surface 10SA of the movable layer 4 becomes easily movable in following relative to the external force (for example, a load from the skin surface) in the direction along the front surface 10SA, which is applied to the front surface side 4S or the back surface side 4B of the movable layer 4.

Specifically, from a viewpoint of allowing the movable layer 4 to move by force smaller than static friction force working between the skin surface SK and the front surface 10SA of the movable layer 4, the number of fused points between the constituent fibers in the movable layer 4 is preferably set in the following range. The number of fused points between the constituent fibers per unit area in the region on the internal side 4M of the movable layer 4 is preferably 70% or less of the number of fused points between the constituent fibers per unit area in either or both of the regions on the front surface side 4S and on the back surface side 4B of the movable layer 4, respectively. The number of fused points is more preferably 65% or less, and further preferably 60% or less. Then, from a viewpoint of securing strength of the nonwoven fabric in the movable layer, the number of fused points is preferably 30% or more, more preferably 35% or more, and further preferably 40% or more. In addition, the strength of the nonwoven fabric is secured, the movable layer 4 becomes hard to be settled and the shape is easily held by setting the number of fused points of the constituent fibers to the above-described lower limit or more.

### [Method for Measuring Fused Point Number]

(i) Preparation of measuring sample:

A measuring sample is prepared by the method shown in (i) Preparation of measuring sample in [Method for measuring moving range of front surface 10SA of nonwoven fabric 10] mentioned above.

(ii) Region on front surface side 4S and back surface side 4B of movable layer 4 of nonwoven fabric 10:

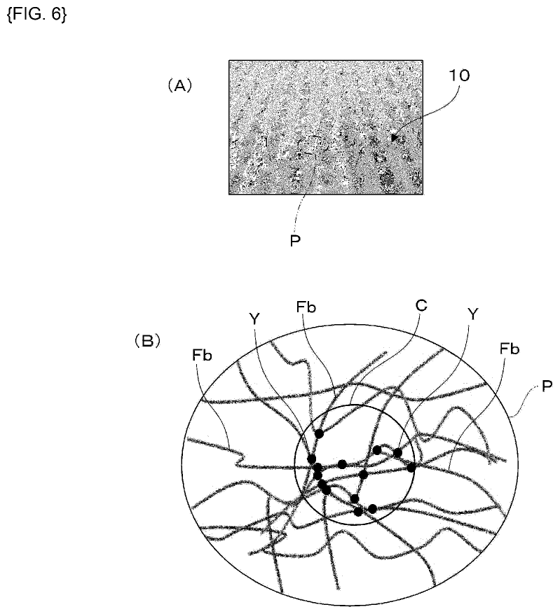

As shown in FIG. 6(A), the nonwoven fabric 10 is observed from the first surface side Z1 and the second surface side Z2 in the state in the plane view at a magnification of 100 times to acquire an observation image of, for example, an observation region P by using a scanning electron microscope (JCM-5100 (trade name) manufactured by JEOL Ltd.).

Subsequently, a reference circle C having a diameter of 0.5 mm (a dimension in the observation image) is placed in the acquired observation image (see FIG. 6(B)), and the fused point number (j) in the reference circle C is counted, and the resultant value is converted into the fused point number (J) per 1 mm² based on the following formula (2).

Fused point number J(piece/mm²)=j×5.1 (2)

In addition, FIG. 6(B) shows the observation image from the first surface side Z1. In this example shown, a black circle part shows a position of the fused point Y in the reference circle C, the number is counted, and the resultant number is taken as a measured value of the fused point number. Numerical values obtained by measuring the number on both sides and converting measured values, respectively, are taken as the numerical values on the front surface side 4S and the back surface side 4B, respectively.

(iii) Region on internal side 4M of movable layer 4 of nonwoven fabric 10:

On the internal side 4M of the movable layer 4 (see FIG. 1), the fused point number is measured on a cross section of the nonwoven fabric in thickness direction (cross section perpendicular to a plane of the nonwoven fabric) in a center portion in thickness direction of the nonwoven fabric 10, and on a cross section perpendicular to the above cross section of the nonwoven fabric in thickness direction in the center portion in thickness direction of the nonwoven fabric 10 in the same method as the observation method using the scanning electron microscope in (ii) described above. Then, a value in the cross section in which the fused point number is larger is adopted as the fused point number per 1 mm² in the region on the internal side 4M of the movable layer 4 of the nonwoven fabric 10.

(iv) With regard to each measurement in (ii) and (iii) described above, observation images in 3 places are arranged for each in the same measuring sample and measured, and an average value is taken as a measured value in each region.

The nonwoven fabric 10 is preferably formed of one sheet of nonwoven fabric, and not a laminated material. Here, the nonwoven fabric means a material after a fiber web is thermally fused, and a material in which the fiber webs are laminated before thermal fusion is defined as one sheet of nonwoven fabric. Whether or not the fiber webs are laminated before thermal fusion can be distinguished by observing the nonwoven fabric by a microscope. In the nonwoven fabric produced, if the fibers melted in a film form are not found, the nonwoven fabric can be defined as “one sheet of nonwoven fabric”. For example, a material having fused points by heat-embossing is deemed as a “material formed by bonding nonwoven fabrics”, and is not one sheet of nonwoven fabric.

The nonwoven fabric 10 is formed of one sheet of nonwoven fabric. Thus, the number of fused points inhibiting the movability in the internal side 4M of the movable layer 4 is decreased, and therefore the movable layer 4 becomes easily movable. For example, a laminated nonwoven fabric has, in the region on an internal side of the laminated nonwoven fabric, fused points at which the fibers are bonded to each other for laminating the nonwoven fabrics, and the fused points work in a direction of inhibiting the above-mentioned movability in plane direction. However, if the nonwoven fabric is formed of one sheet, such a nonwoven fabric requires no interlayer fused points as in the laminated nonwoven fabric, and therefore the movable layer 4 becomes easily movable. Therefore, the movable zone in the movable layer 4 is widened.

In the nonwoven fabric 10, the number of constituent fibers per unit area in the region on the internal side 4M of the movable layer 4 (see FIG. 1) is preferably less than the number of constituent fibers per unit area in either or both of the regions on the front surface side 4S and on the back surface side 4B of the movable layer 4, respectively. Thus, the region on the internal side 4M of the movable layer 4 further secures a distance between the fibers and becomes further easily movable than the region on the front surface side 4S or the back surface side 4B of the movable layer 4.

Specifically, the number of constituent fibers per unit area in the region on the internal side 4M of the movable layer 4 (see FIG. 1) is preferably 80% or less, more preferably 75% or less, and further preferably 70% or less of the number of constituent fibers per unit area in either or both of the regions on the front surface side 4S and on the back surface side 4B of the movable layer 4, respectively. Then, from a viewpoint of securing the strength of the nonwoven fabric in the movable layer, the number is preferably 40% or more, more preferably 45% or more, and further preferably 50% or more.

The movability of the region on the internal side 4M of the movable layer is increased by forming a configuration of the number of constituent fibers per unit area as described above. In addition, cushioning properties of the movable layer 4 are easily obtained by setting the number of constituent fibers to the above-described lower limit or more.

### [Method for Measuring the Number of Fibers]

(i) Preparation of measuring sample:

A measuring sample is prepared by the method shown in (i) Preparation of measuring sample in [Method for measuring moving range of front surface 10SA of nonwoven fabric 10] mentioned above.

(ii) Region on front surface side 4S and back surface side 4B of movable layer 4 of nonwoven fabric 10:

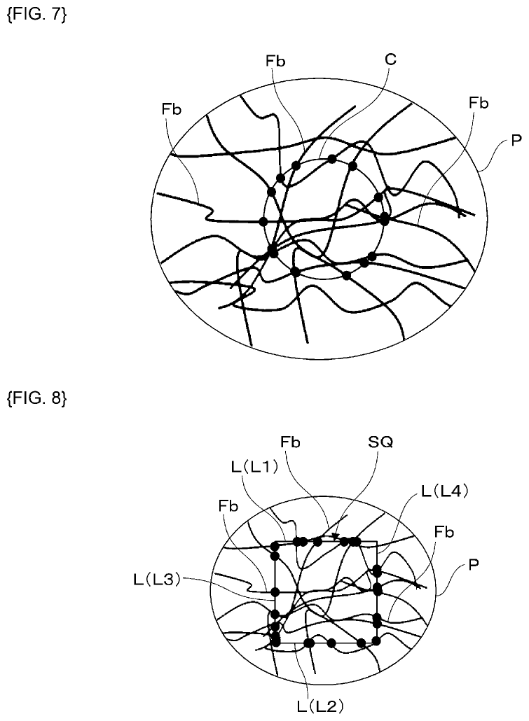

In the same manner as (ii) in [Method for measuring fused point number] mentioned above, observation images from the first surface side Z1 and the second surface side Z2 are acquired (for example, the observation image shown by a sign P in FIG. 7). A reference circle C shown in the above-mentioned FIG. 6 is placed onto each observation image (see FIG. 7). The number of fibers Fb passing through a line of the reference circle C is counted, and a half of a sum of the number is taken as the number of fibers (n) existing in the area, and the resultant value is converted into the number of fibers per 1 mm² (N) based on the following formula (3). In addition, FIG. 7 shows the observation image from the first surface side Z1. In this example shown, a black circle part is a position in which the fiber Fb passes through the reference circle C, and the number thereof is counted, and the resultant value is converted.

The number of fibers N(fibers/mm²)=(n/2)×5.1 (3)

(iii) Region on internal side 4M of movable layer 4 of nonwoven fabric 10:

In the same manner as (iii) in [Method for measuring fused point number] described above, observation images are acquired on a cross section of the nonwoven fabric (cross section perpendicular to a plane of the nonwoven fabric plane) in thickness direction in a center portion in thickness direction of the nonwoven fabric 10, and on a cross section perpendicular to the above cross section of the nonwoven fabric in thickness direction in the center portion in thickness direction of the nonwoven fabric 10, and the number of fibers is measured by using the same method as the observation method using the scanning electron microscope in (ii) described above. Then, a value in the cross section in which the number of fibers is larger is adopted as the number of fibers in the region on the internal side 4M of the movable layer 4 of the nonwoven fabric 10.

In addition, when the nonwoven fabric 10 has the concavo-convex portion, in the region on the internal side 4M of the movable layer 4 of the nonwoven fabric 10, the number is measured on a cross section passing through a center in thickness direction of the wall portion 3 of the concavo-convex portion and along thickness direction of the wall portion 3 perpendicular to the wall portion 3, and on a cross section along the wall portion perpendicular to the above cross section, for example.

(iv) With regard to each measurement in (ii) and (iii) described above, observation images in 3 places are arranged for each in the same sample and measured, and an average value is taken as a measured value.

When the fibers constituting the nonwoven fabric is vertical to plane direction in the plane view of the nonwoven fabric, the fibers can move in a falling over manner. Therefore, from a viewpoint of facilitating the movability of the fibers with each other, a fiber orientation parameter in the region on the internal side 4M of the movable layer is preferably 1.1 times or more to the fiber orientation parameter in either or both of the regions on the front surface side 4S and on the back surface side 4B of the movable layer 4 (see FIG. 1). The fiber orientation parameter is more preferably 1.15 times or more, and further preferably 1.2 times or more. Then, from a viewpoint of securing the strength of the nonwoven fabric in the movable layer, the fiber orientation parameter is preferably 1.4 times or less, more preferably 1.35 times or less, and further preferably 1.3 times or less.

The internal side 4M of the movable layer becomes easily movable in the direction along the front surface 10SA by having the above-described relationship. That is, the moving range in the movable layer 4 is widened. In addition, the movable layer 4 has sufficient movability by setting the fiber orientation parameter to the above-described upper limit or less. On the other hand, strength of the movable layer 4 in thickness direction can be sufficiently secured by setting the fiber orientation parameter to the above-described lower limit or more. Therefore, the movable layer becomes hard to be flattened even by a load in thickness direction, the movable zone in the movable layer 4 is secured, the nonwoven fabric 10 easily follows the movement in the direction along the surface of the skin surface SK, and the chafing onto the skin surface becomes hard to occur.

In addition, the fiber orientation parameter is a numerical value shown in <Definition of fiber orientation parameter> described below, and is measured by [Method for measuring fiber orientation parameter] described below.

### <Definition of Fiber Orientation Parameter>

A degree at which the fibers are aligned in one direction is taken as the fiber orientation parameter, and on the front surface side 4S or the back surface side 4B of the movable layer 4, the degree at which the fibers are oriented in a direction (for example, an MD direction or a CD direction) in the state of the plane view is measured based on the method for measuring the fiber orientation parameter. The fiber orientation parameter on the internal side 4M of the movable layer is taken as the degree at which the fibers are oriented in a vertical direction or a horizontal direction in the cross section in thickness direction. Here, the MD direction is a machine direction, and the CD direction is a cross direction perpendicular to the MD direction.

The fiber orientation parameter on the internal side 4M of the movable layer is higher than the fiber orientation parameter on the front surface side 4S or the back surface side 4B, and therefore the internal side 4M of the movable layer becomes further easily movable in the direction along the front surface. Therefore, the moving range in the movable layer 4 is widened.

### [Method for Measuring Fiber Orientation Parameter]

(i) Preparation of measuring sample:

A measuring sample is prepared by the method shown in (i) Preparation of measuring sample in [Method for measuring moving range of front surface 10SA of nonwoven fabric 10] mentioned above.

(ii) Region on front surface side 4S and back surface side 4B of movable layer 4 of nonwoven fabric 10:

In the same manner as (ii) in [Method for measuring fused point number] mentioned above, observation images from the first surface side Z1 and the second surface side Z2 are acquired (for example, the observation image shown by a sign P in FIG. 8). A reference line L forming a square SQ having 0.5 mm×0.5 mm (a dimension in the observation image) is placed onto each observation image (see FIG. 8). Here, the reference line L is created so as to coincide with a longitudinal direction (for example, the MD direction) of the nonwoven fabric or an article into which the nonwoven fabric is assembled, or a direction (for example, the CD direction) perpendicular to the longitudinal direction. That is, upper and lower reference lines are formed of an upper side L1 and a lower side L2 of the square SQ, the fibers passing through the upper and lower reference lines are taken as “the number of upper and lower fibers”, left and right reference lines are formed of a left side L3 and a right side L4 of the square, and the fibers passing through the left and right reference lines are taken as “the number of left and right fibers”.

A larger value of the number of upper and lower fibers and the number of left and right fibers is taken as A and a smaller value thereof is taken as B, and the fiber orientation parameter (K) is calculated based on the following formula (4).

Fiber orientation parameter K(degree)=[A/(A+B)]×100 (4)

In addition, FIG. 7 shows the observation image from the first surface side Z1. In this example shown, a black circle part is a position in which a fiber Fb passes through each side (reference line) of the square.

(iii) Region on internal side 4M of movable layer 4 of nonwoven fabric 10:

On the internal side 4M of the movable layer, the fiber orientation parameter is measured on the cross section of the nonwoven fabric in thickness direction (cross section perpendicular to a plane of the nonwoven fabric) in the center portion in thickness direction of the nonwoven fabric 10, by using the same method as the observation method using the scanning electron microscope in (ii) described above.

(iv) With regard to each measurement in (ii) and (iii) described above, observation images in 3 places are arranged for each in the same sample and measured, and an average value is taken as a measured value.

In the nonwoven fabric 10, a relationship between the region on the internal side 4M of the movable layer 4, and the regions on the front surface side 4S and the back surface side 4B of the movable layer 4 preferably satisfies at least one, more preferably satisfies two or more, and particularly preferably satisfies all of the preferable numerical ranges of the fused point of fibers, the number of fibers and the fiber orientation parameter as mentioned above. When the relationship satisfies all thereof, a state of becoming zero friction (no skin following) between the skin surface and the front surface 10SA (region on the front surface side 4S of the movable layer 4) of the nonwoven fabric 10 can be most strongly eliminated, and a chafing suppression effect of the nonwoven fabric onto the skin surface is further easily produced.

Next, more specific structure of the nonwoven fabric 10A shown in FIGS. 3 to 5 is described.

In the nonwoven fabric 10A, the outer surface fiber layer 1 on the first surface side Z1 has the first and second outer surface fiber layers 11 and 12 in the region on the front surface side 4S of the movable layer 4. The first and second outer surface fiber layers 11 and 12 have a length extending along each of different directions which cross each other in a plane view of the nonwoven fabric 10A. The extending directions are an X direction and a Y direction perpendicular to each other along a side of the nonwoven fabric 10A. As an example, the Y direction is a longitudinal direction of the nonwoven fabric 10A, and the X direction is a crosswise direction of the nonwoven fabric 10A.

The first outer surface fiber layer 11 is continued and extended without a break in the Y direction in the plane view of the nonwoven fabric 10A. Namely, the first outer surface fiber layer 11 is continued without a break in the longitudinal direction as a whole of the nonwoven fabric 10A, and a plurality thereof is arranged at intervals to the X direction perpendicular to the Y direction.

The second outer surface fiber layer 12 is extended in the X direction and is arranged by linking the first outer surface fiber layers 11 and 11 separated from each other and arranged in parallel in the X direction. By “linking the first outer surface fiber layers 11 and 11” is meant that the second outer surface fiber layers 12 adjacent to each other by interposing the first outer surface fiber layer 11 are aligned in a linear form. Specifically, the expression means that a deviation of a crosswise center line of the second outer surface fiber layer 12 extending in the X direction from a crosswise center line of the second outer surface fiber layer 12 extending in the X direction, adjacent to each other by interposing the first outer surface fiber layer 11, is within the range of a width (length in the Y direction) of the second outer surface fiber layer 12, for example, within 5 mm.

In the second outer surface fiber layer 12, a position on the first surface side Z1 is preferably formed to be somewhat lower than a position of the first outer surface fiber layer 11. Therefore, in the second outer surface fiber layer 12, a length in the X direction is divided by interposition of the first outer surface fiber layer 11, and the plurality thereof form rows in the X direction by being separated from each other. Moreover, a width (length in the Y direction) of the second outer surface fiber layer 12 is preferably formed to be narrower than a width (length in the X direction) of the first outer surface fiber layer 11. With regard to rows of the second outer surface fiber layers 12 in the X direction, a plurality thereof is further arranged in the Y direction by being separated from each other. In addition, a shape of the second outer surface fiber layer 12 is not limited to a shape in the embodiment, for example, the position and the width thereof on the first surface side Z1 may be set to the same as the position and the width on the first outer surface fiber layer 11.

When the outer surface fiber layer 1 has a plurality of kinds different in the extending directions, as described above, an expression “different directions which cross each other in a plane view” deemed as the extending direction is not limited to the X direction and the Y direction. The expression may take various forms as long as the expression refers to crossing directions in plane direction (direction in parallel to a direction along the front surface) of the nonwoven fabric 10.

The outer surface fiber layer 2 on the second surface side Z2 is in a region of the back surface side 4B of the movable layer 4, and a plurality thereof is arranged at intervals. Specifically, the outer surface fiber layers 2 on the second surface side Z2 cover separation space between the first outer surface fiber layers 11 and 11 on the first surface side Z1, and the plurality thereof is separated from each other and arranged in rows along the extending direction (Y direction) of the outer surface fiber layer 11. Further, with regard to rows of the outer surface fiber layers 2 in the Y direction, a plurality thereof is arranged at intervals in the X direction perpendicular to the Y direction. That is, the outer surface fiber layer 2 is also arrayed in the X direction. An array direction of the outer surface fiber layers 2 coincides with an extending direction of the outer surface fiber layer 1 in a position in which the outer surface fiber layer 2 is not overlapped with the outer surface fiber layer 1 in the plane view. Therefore, when the extending direction of the outer surface fiber layer 1 takes a direction different from the above-described X direction and the above-described Y direction, the array direction of the outer surface fiber layer 2 also takes the direction different from the above-described X direction and the above-described Y direction corresponding thereto.

Moreover, the wall portion 3 is in a region of the internal side 4M of the movable layer 4, and has a first wall portion 31 linking the first outer surface fiber layer 11 on the first surface side Z1 with the outer surface fiber layer 2 on the second surface side Z2, and a second wall portion 32 linking the second outer surface fiber layer 12 on the first surface side Z1 with the outer surface fiber layer 2 on the second surface side Z2. With regard to the wall portions 3 (the first wall portions 31 and the second wall portions 32), a plurality thereof is arranged at intervals in plane direction of the nonwoven fabric 10 according to separation arrangement of the outer surface fiber layers 1 and 2.

A plurality of the wall portions 3 constituting the first wall portions 31 and the second wall portions 32 is arranged along different directions which cross each other in a plane view in the nonwoven fabric 10. Specifically, the first wall portions 31 have a length which coincides with the side of the outer surface fiber layer 2 on the second surface side Z2 in the Y direction and has the surface along the extending direction of the first outer surface fiber layer 11 on the first surface side Z1. That is, the surface of the first wall portion 31 is arranged along the Y direction. On the other hand, the second wall portions 32 have a length which coincides with the side of the outer surface fiber layer 2 on the second surface side Z2 in the X direction and has the surface along the extending direction of the second outer surface fiber layer 12 on the first surface side Z1. That is, the surface of the second wall portion 32 is arranged along the X direction. A direction along which the surface of the wall portions 3 (first wall portions 31 and second wall portions 32) is arranged coincides with the extending direction of the outer surface fiber layer 1 (first outer surface fiber layer 11 and second outer surface fiber layer 12). Therefore, when the extending direction of the outer surface fiber layer 1 takes a direction different from the above-described X direction and the above-described Y direction, the direction along which the surface of the wall portion 3 is arranged also takes a direction different from the above-described X direction and the above-described Y direction corresponding thereto.

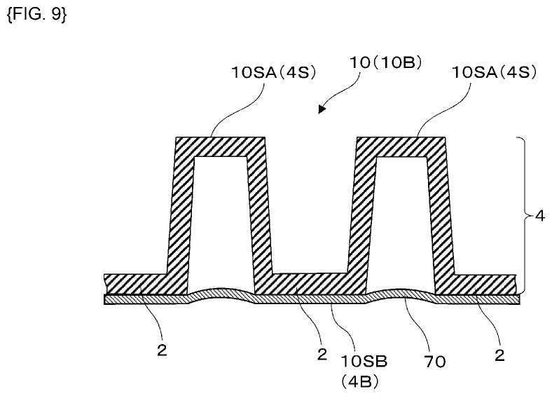

Next, another preferred embodiment will be explained below, referring to FIG. 9. In addition, the same sign is placed to the same component as the component in the nonwoven fabric 10A according to the above-described embodiment shown in FIGS. 3 to 5.

In the nonwoven fabric 10(10B) shown in FIG. 9, a coating layer 70 is arranged on a whole surface on the second surface side Z2 of the above-mentioned nonwoven fabric 10A. A configuration except the coating layer 70 is the same as configuration of the above-described nonwoven fabric 10A. The coating layer 70 is positioned in the region on the back surface side 4B of the movable layer 4. In this nonwoven fabric 10B, when the front surface 10SA (region on the front surface side 4S of the movable layer 4) follows the skin surface SK, the coating layer 70 being the back surface 10SB (region on the back surface side 4B of the movable layer 4) does not slide, and the front surface 10SA becomes easily movable in the direction along the front surface 10SA.

The above-described nonwoven fabric 10 preferably satisfies the following conditions.

A basis weight amount is different in the nonwoven fabric 10, and with regard to thickness direction (Z direction) of the movable layer 4, the nonwoven fabric 10 preferably has, in the region on the internal side 4M of the movable layer 4, a region in which the basis weight amount is smaller than the amount in either or both of the regions on the front surface side 4S and on the back surface side 4B, respectively. In this part in which the basis weight amount is small, a space between the fibers is wide, and therefore the movement in the direction along the front surface 10SA becomes easy.

From a viewpoint of securing a movable space between the fibers, the apparent thickness of the nonwoven fabric 10 is preferably 1.5 mm or more, more preferably 2 mm or more, and further preferably 3 mm or more. Then, an upper limit of the apparent thickness is not particularly limited, but from a viewpoint of providing the absorbent article with excellent portability or the like in the form of article of commerce such as the absorbent article, the upper limit is preferably 10 mm or less, more preferably 9 mm or less, and further preferably 8 mm or less.

### [Method for Measuring Apparent Thickness of Nonwoven Fabric]

A measuring object nonwoven fabric is cut to 10 cm×10 cm to prepare a measuring sample. When the size is unable to be taken, the nonwoven fabric is cut in an area as large as possible to prepare prepared a measuring sample. A thickness at a load of 50 Pa is measured by using a laser displacement sensor head (high-precision displacement sensor ZS-LD80, manufactured by OMRON Corporation). Measurement is carried out in three places, and an average value is taken as the apparent thickness.

### [Method for Measuring Basis Weight Amount of Nonwoven Fabric]

A measuring sample is prepared in the same manner as the method for measuring apparent thickness described above. Mass of the measuring sample is measured to the second decimal place in a g unit by using a balance, and a value obtained by dividing the measured value by an area of the measuring sample is taken as the basis weight amount.

In a method for measuring basis weight of each site in the nonwoven fabric, each site is cut out from a measuring object nonwoven fabric, and a width and a length of the cut-out object are accurately measured to the first decimal place in a mm unit. Then, a measuring sample is cut out until a total reaches 50 mm² or more, and mass of the measuring sample in which the total reaches 50 mm² or more is measured to the fourth decimal place in a g unit by using a precision balance, and a value obtained by dividing the measured value by an area of the measuring sample is taken as the basis weight amount.

In the nonwoven fabric 10, the constituent fibers have a sheath-core structure, and a sheath-core ratio of the fibers in the sheath-core structure is preferably different in the nonwoven fabric 10. Then, with regard to thickness direction of the movable layer 4, the nonwoven fabric 10 preferably has, in the region on the internal side 4M of the movable layer 4, a region in which a sheath ratio is smaller than the ratio in either or both of the regions on the front surface side 4S and on the back surface side 4B, respectively. The sheath-core ratio is defined by a mass ratio (% by mass) of a core resin amount to a sheath resin amount during preparing the fibers. In a place in which the sheath ratio is small, a fused resin amount between the fibers is small, and therefore a fused part is easily deformed, and the place is formed into an easily movable structure.

In the case where the fibers have a sheath-core structure, different resins can be used for the core component and the sheath component. Among these, from a viewpoint of effectively fusing the fibers to each other, conjugate fibers comprising a low-melting component and a high-melting component (for example, sheath-core type conjugate fibers in which the sheath is low-melting component and the core is high-melting component) are preferably used. Specific examples of the sheath-core type conjugate fibers in which the sheath is low-melting component and the core is high-melting component include sheath-core type conjugate fibers in which the sheath is polyethylene (PE) and the core is polyethylene terephthalate (PET).

Further, in the sheath-core type conjugate fibers, in the case where the sheath resin component has lower glass transition temperature than the core resin component (hereinafter, referred to as low glass transition temperature resin) (for example, the core resin component is PET and the sheath resin component is PE), recoverability of thickness of the nonwoven fabric can be enhanced by reducing the mass ratio of the low glass transition temperature resin component. As factors that contribute to this situation, the following factors can be considered. It is known that a low glass transition temperature resin has low relaxation modulus. Moreover, it is also known that recover to the deformation is hard to occur when the relaxation modulus is low. Therefore it is considered that higher thickness recoverability can be provided to the nonwoven fabric by reducing the low glass transition temperature resin component as much as possible.

In the case of the sheath-core type conjugate fibers, a proportion of the low glass transition temperature resin component (PE and the like) with respect to the total fiber mass is preferably smaller than a proportion of the resin component having high glass transition temperature (PET and the like) with respect to the total fiber mass, by mass ratio. Specifically, the proportion of the low glass transition temperature resin component with respect to the total fiber mass is preferably 45 mass % or less, and more preferably 40 mass % or less, by mass ratio. Thickness recoverability of the nonwoven fabric can be enhanced by reducing the proportion of the low glass transition temperature resin component. Moreover, from the viewpoint of producing the nonwoven fabric, the proportion is preferably 10 mass % or more, and more preferably 20 mass % or more, by mass ratio.

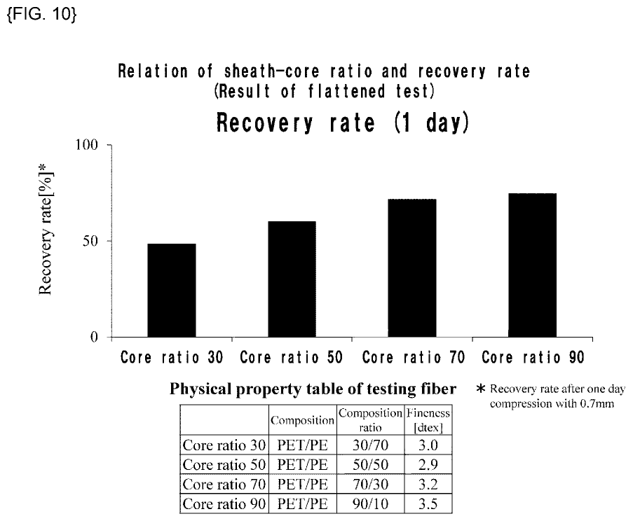

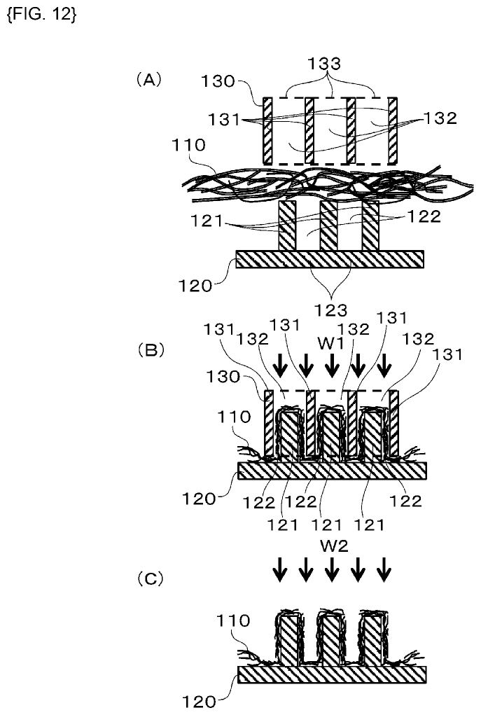

This can also be seen from a graph shown in FIG. 10. FIG. 10 shows recoverability after one day compression of a nonwoven fabric in the case of changing the proportion of the core resin component (PET) and the sheath resin component (PE) (measuring method is based on the method shown in the “recoverability after one day compression” shown in Examples described below). In addition, the nonwoven fabric was prepared according to an air-through production method including a step shown in FIG. 12. Blowing treatment by first hot air W1 was applied thereto under conditions of a temperature of 160° C., an air speed of 54 m/s and a blowing time of 6 s. Blowing treatment by second hot air was applied thereto under conditions of a temperature of 160° C., an air speed of 6 m/s and a blowing time of 6 s. The apparent thickness of the prepared nonwoven fabric was 6.0 mm for a type of “core ratio of 30”, 6.9 mm for a type of “core ratio of 50”, 6.6 mm for a type of “core ratio of 70”, and 6.0 mm for a type of “core ratio of 90”. As the proportion of the sheath resin component being PE having low glass transition temperature is smaller (the proportion of the core resin component is larger), the recoverability after one day compression is higher. In particular, when the proportion of the sheath resin component becomes less than 50 mass % (the proportion of the core resin component becomes more than 50 mass %), the recoverability after one day compression becomes 70% or more, and such a case is preferable.

In the nonwoven fabric 10, the number of crimped fibers per unit area in the nonwoven fabric is different. Then, with regard to thickness direction of the movable layer 4, the nonwoven fabric 10 preferably has, in the region on the internal side 4M of the movable layer 4, a region in which the number of crimped fibers is smaller than the number in either or both of the regions on the front surface side 4S and on the back surface side 4B, respectively. In this place in which the number of crimped fibers is small, entanglement of the fibers is hard to occur, and therefore a case where the fibers are entangled and the movement is inhibited is hardly induced, and the resultant product becomes easily movable. Specifically, the nonwoven fabric 10 preferably has the region in which the number of crimped fibers is small in part of thickness direction of the movable layer 4. For example, the nonwoven fabric 10 preferably has the region in which the number of crimped fibers is small in a partial region of the height direction of the wall portion 3.

Alternatively, the wall portion 3 as a whole may be formed into the region in which the number of crimped fibers is small.

In the nonwoven fabric 10, a fiber diameter of the constituent fibers is different. With regard to thickness direction of the movable layer 4, the nonwoven fabric 10 preferably has, in the region on the internal side 4M of the movable layer 4, a region in which the fiber diameter is larger than the diameter in either or both of the regions on the front surface side 4S and on the back surface side 4B, respectively. Specifically, the nonwoven fabric 10 preferably has the region in which the fiber diameter is large in a partial region of the height direction of the wall portion 3. In the region in which the fiber diameter is large, the fibers are not densely packed, and therefore entanglement of the fibers is hard to occur, and a case where the fibers are entangled and the movement is inhibited is not induced, and the resultant product becomes easily movable.

In the nonwoven fabric 10, ratio of thermal expansion and contraction of the constituent fibers is different, and with regard to thickness direction of the movable layer 4, the nonwoven fabric 10 preferably has, in the region on the internal side 4M of the movable layer 4, a region in which the fibers are further thermally stretchable than a level in either or both of the regions on the front surface side 4S and on the back surface side 4B, respectively. For example, the nonwoven fabric 10 preferably has the region in which the fibers are thermally stretchable in thickness direction of the movable layer 4. In this region in which the fibers are thermally stretchable, a convex portion height is increased and the apparent thickness is increased, and therefore the moving range of the front surface 10SA is increased, as shown in the following formula. When a moving length of a front surface 10SA is taken as D, an apparent thickness is taken as t and an external angle is taken as e, the relationship according to the above-mentioned formula (1) is obtained. Specifically, the nonwoven fabric 10 preferably has, in the wall portion 3, the region in which the fibers are thermally stretchable.

Alternatively, the wall portion 3 as a whole may be formed into a fiber region in which the fibers are thermally stretchable.

Further, although not shown, in the nonwoven fabric 10, the outer surface fiber layers 1, 2 and the wall portion 3 are formed with each other, in which at least part of the fibers are fused to each other, and united without seam. In the nonwoven fabric 10, the wall portion 3 connects the outer surface fiber layer 1 on the first surface side Z1 and the outer surface fiber layer 2 on the second surface side Z2 to support both. Thus, the nonwoven fabric 10 is formed into a bulky and thick material. Thickness of the nonwoven fabric 10 means the apparent thickness in a shaped form of the nonwoven fabric as a whole, and not a thickness in a local of the outer surface fiber layers 1, 2 or the wall portion 3.

In addition, in the nonwoven fabric 10, also in each site other than the outer surface fiber layers 1, 2, the wall portion 3 and a connecting part, the fibers are fused at intersections of at least part of the fibers with each other. Further, the nonwoven fabric 10 may have intersections in which the fibers are not fused. Furthermore, the nonwoven fabric 10 may contain fibers other than the thermoplastic fibers, including a case where the thermoplastic fibers are fused at intersections with the fibers other than the thermoplastic fibers.

As mentioned above, the nonwoven fabric according to the present invention is not limited to a material having the above-described shape and can take various shapes, as long as the front surface has the movable zone of 5 mm or more in the direction along the front surface.

In addition to the matters described above, for example, the nonwoven fabric having the flat surface without having concavo-convex shape on the front surface or the back surface can also be applied as the nonwoven fabric having the movable zone of 5 mm or more according to the present invention by having the region on the internal side 4M of the movable layer 4. The region on the internal side 4M of the movable layer 4 preferably satisfies conditions of the number of fused points, the number of fibers, the fiber orientation parameter, the basis weight amount, the sheath-core ratio, the number of crimped fibers, the thermally stretchable region and the like as described above. Moreover, the nonwoven fabric having the configuration as shown in FIG. 1 in JP-A-2012- and the nonwoven fabric having the configuration as shown in FIG. 1 in JP-A-2016-79529 can also be applied as the nonwoven fabric according to the present invention by properly setting the respective conditions of the number of fused points described above, the external angle of the wall portion, and the like.

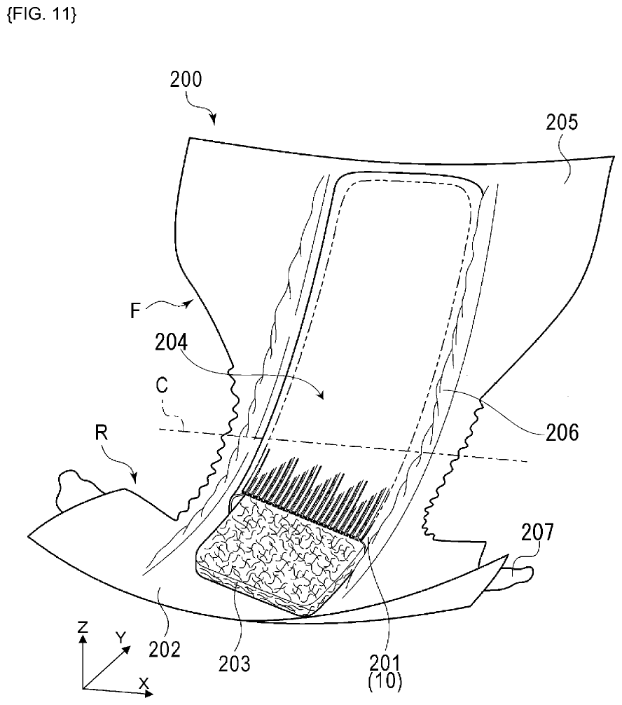

Next, referring to FIG. 11, as one preferred embodiment of the absorbent article in which the nonwoven fabric according to the present invention is used for the topsheet, an example of application to a main body 4 of a disposable diaper 200 is described below. The disposable diaper shown in the figure is a tape-type disposable diaper for infants, and is shown in a state in which the diaper in a flatly unfolded state is a little bent and is viewed from the inside (the skin-contact surface side).

As shown in FIG. 11, an absorbent main body 204 used in a diaper 200 according to the present invention has the following basic configuration. Accordingly, the diaper 200 has a fluid-permeable topsheet 201 disposed on the skin-contact surface side, a fluid-hardly-permeable backsheet 202 disposed on the skin non-contact surface side, and a fluid-retainable absorbent body 203 interposed and arranged between the topsheet 201 and the backsheet 202.

The nonwoven fabric 10 of the above-described embodiment is applied to the topsheet 201. In the topsheet 201, the nonwoven fabric 10A shown in FIG. 3 is disposed by directing the first surface side Z1 toward a skin contact surface side. The backsheet 202, in the unfolded state, has a shape with its both side edges being constricted inside at the central portion C in the longitudinal direction, and may have a single sheet or a plurality of sheets. In the example, lateral leakage prevention gathers 206 formed by the side sheets 205 are provided. In addition, in FIG. 11, the arrangement and boundaries of each component are not strictly shown, and a structure thereof is not limited as long as it is a common form for nappies of this type.

The above diaper 200 is of a tape type, and a fastening tape 207 is provided at a flap portion on the rear side R. The fastening tape 207 is attached to a tape attachment portion (not shown) provided on the flap portion on the abdomen side F, whereby the diaper can be worn and fixed. At this time, the central portion C of the diaper is gently bent inwards, with the absorbent body 203 extending from the hip portion to the lower abdomen. The followability of the surface of the nonwoven fabric to the movement of the skin surface is measured, and the diaper can exhibit soft texture and flexible texture by applying the nonwoven fabric 10 as the topsheet 1.

As a shape of an absorbent body 204, the absorbent body 204 has a vertically long shape having the longitudinal direction to be arranged from a lower abdomen side to a hip side through a wearer's crotch part during wearing and the crosswise direction perpendicular thereto. In the specification, a direction having a relatively large length in the plane view of the absorbent main body 204 is referred to as the longitudinal direction, and a direction perpendicular to the longitudinal direction is referred to as the crosswise direction. The above-described longitudinal direction typically coincides with a front-back direction of a human body in a worn state.

It is preferable that the topsheet 201 is formed of the above-mentioned nonwoven fabric 10 according to the present invention, and is a hydrophilic nonwoven fabric. As the hydrophilic nonwoven fabric, fibers which are subjected to hydrophillisation treatment, such as conjugate fibers of polypropylene and polyethylene, and conjugate fibers of polyethylene terephthalate and polyethylene or the like, can be preferably used.

As the backsheet 202 and the absorbent body 203, for example, materials described in JP-A-2013-147784 and JP-A-2014-005565 can be used.

As the topsheet 201 of the diaper 200, in the nonwoven fabric 10 according to the present invention, the movable layer 4 is movable by 5 mm or more in the direction along the front surface, and therefore the topsheet 201 becomes easy to follow the movement of wearer's buttocks. Therefore, chafing onto the skin surface by the topsheet 201 is suppressed and the topsheet 201 serves as the skin surface-friendly topsheet. Further, the topsheet 201 always matches an excretion point and serves as an excellent material in which leak is suppressed. Furthermore, the topsheet can be always placed in a desired portion, and therefore can also have the area smaller than ever before.

The nonwoven fabric according to the present invention can be used for a variety of uses. For example, the nonwoven fabric can be suitably used as the topsheet of the absorbent article such as a disposable diaper for an adult or for an infant, a sanitary napkin, a panty liner, a urine pad and the like. Further, the nonwoven fabric can be also used in the form of a sublayer to be interposed between a topsheet and an absorbent body of a sanitary item, a diaper or the like, a covering sheet (core-wrapping sheet) of the absorbent body, or the like. Furthermore, the nonwoven fabric can be also used in a wiping sheet for cleaning.

Next, one preferred embodiment of a method for producing the nonwoven fabric 10 of the embodiment is described below, referring to FIG. 12.

In the method for producing the nonwoven fabric 10 in the embodiment, a support male material 120 and a support female material 130 for shaping a fiber web 110 before being processed into the nonwoven fabric are used. As shown in FIG. 12(A), the fiber web 110 is placed on the support male material 120 and pushed, and interposed with the support female material 130 from above to shape the fiber web 110.

The support male material 120 has a plurality of projections 121 in corresponding to the four wall portions 3 surrounding the space portion of the nonwoven fabric 10 and a position in which the outer surface fiber layer 2 on the second surface side Z2 (see FIG. 3 and the like) is shaped. A place between the projections 121 and 121 is formed into a concave portion 122 corresponding to the position in which the outer surface fiber layer 1 on the first surface side Z1 is shaped. Thus, the support male material 120 has a concavo-convex shape, and the projections 121 and the concave portions 122 are alternately arranged in different directions in plane view. A bottom portion 123 of the concave portion 122 has a structure through which hot air is blown, and a plurality of holes is arranged (not shown), for example.

The support female material 130 has a lattice-shaped projection 131 in corresponding to the concave portions 122 of the support male material 120. A place between the projections 131 and 131 is formed into a concave portion 132 corresponding to the projection 121 of the support male material 120. Thus, the support female material 130 has a concavo-convex shape, and the projections 131 and the concave portions 122 are alternately arranged in different directions in plane view. A bottom portion 133 of the concave portion 132 has a structure through which hot air is blown, and a plurality of holes is arranged, for example. A distance between the projections 131 and 131 is formed to be wider than a width of the projection 121 of the support male material 120. The distance is appropriately set so as to preferably shape the wall portions 3 in which the fibers are oriented in thickness direction by interposing the fiber web 110 with the projection 121 of the support male material 120 and the projection 131 of the support female material 130.

Moreover, arrangement of projections 131 in the support female material 130 may have not only the lattice shape, but also other patterns. For example, the arrangement of projections 131 may have a pattern in which the support female material 130 coincides with the support concave portions 122 of the support male material 120 and has projections 131 being continued in one direction in the plane view, although the pattern is not shown in FIGs. In this case, a place between the projections 131 and 131 is formed into a support concave portion 132, in corresponding to the projection 121 of the support male material 120 and being continued in the one direction. Thus, the support female material 130 has a concavo-convex shape, and the projections 131 and the support concave portions 132 are alternately arranged in a direction perpendicular to the one direction. Specific examples thereof include a pattern of drum shape in which a plurality of ring-shaped disk is linked in a rotation axis direction at equal intervals. In this case, the height of convex portion 82 extending in X direction shown in FIG. 3 is formed to be lower, in comparison with the case of a pattern in which the support female material 130 arranges the projections 131 in a lattice shape.

First, in the embodiment, fiber web 110 before being fused is supplied from a carding machine (not shown) to an apparatus for shaping the web so as to have a predetermined thickness.

Next, as shown in FIG. 12(A), the fiber web 110 containing the thermoplastic fibers is arranged on the support male material 120, and the projections 121 of the support male material 120 are inserted into the support concave portions 132 of the support female material 130 from above the fiber web 110. Moreover, the projections 131 of the support female material 130 are inserted into the support concave portions 122 of the support male material 120. Thus, the fibers are oriented in thickness direction and plane direction.

As shown in FIG. 12(B), in this state, first hot air W1 is blown from a side of the support female material 130 to the fiber web 110. Thus, in the fiber web 110, the fibers are fused at a degree capable of keeping the concavo-convex shape of the nonwoven fabric 10.

Between a top portion of the projection 121 and a bottom portion of the concave portion 132, blowing of the first hot air W1 is suppressed, and the fibers are fused with each other in plane direction. Thus, a fiber layer corresponding to the outer surface fiber layer 2 on the second surface side Z2 is shaped. Moreover, between a bottom portion of the concave portion 122 and a top portion of a protruding portion 131, the fibers are oriented in plane direction. The protruding portion 131 inhibits hot air, and therefore the formed fiber layer has a small amount of fusion, and a smooth fiber layer is realized. Thus, the fiber layer corresponding to the outer surface fiber layer 1 on the first surface side Z1 is shaped. At this time, a shape of the wall portions 3 in which the fibers are oriented in thickness direction is kept.

In addition, arrows in the drawing schematically show the flow of the first hot air W1.

A temperature of the first hot air W1 is preferably set to a temperature at which the shape having vertical orientation of the thermoplastic fibers can be kept. The temperature of the first hot air W1 is preferably higher by 0° C. or more and 70° C. or less than the melting point of a thermoplastic fiber constituting the fiber web 110 with consideration of common fiber materials used for products of this type.

From a viewpoint of effectively fusing the fibers, an air speed of the first hot air W1 is preferably 2 m/s or more.

Thus, the fiber web 110 is temporarily fused for keeping the concavo-convex shape.

Next, the support female material 130 is removed. Then, as shown in FIG. 12(C), second hot air W2 at a temperature at which each fiber in the fiber web 110 shaped in the concavo-convex shape can be properly fused is blown to further fuse the fibers with each other. In this case, the second hot air W2 is preferably also blown, in the same manner as the first hot air W1, from the second surface side Z2 in the nonwoven fabric 10 onto the fiber web 110. A temperature of the second hot air W2 is preferably higher by 0° C. or more and 70° C. or less than the melting point of thermoplastic fibers constituting the fiber web 110 with consideration of common fiber materials used for products of this type.

The air speed of the second hot air W2 is, although the setup depends on the height of the projection 121 of the support male material 120, preferably 3 m/s or more. Thus, satisfactory heat transfer to the fibers is achieved to fuse the fibers with each other, and satisfactory fixing of the concavo-convex shape can be achieved.

As the thermoplastic fibers, the thermoplastic fibers ordinarily used in a raw material of the nonwoven fabric can be adopted without particular restriction, and specific examples thereof include fibers comprising single resin component and conjugate fibers such as a sheath-core type and a side-by-side type.

As described above, the nonwoven fabric 10 is manufactured.

In the nonwoven fabric 10 obtained, the second surface side Z2 is a side to which the first hot air W1 and the second hot air W2 are blown, and therefore the number of fused points between the fibers in the outer surface fiber layer 2 on the second surface side Z2 becomes large. Thus, a difference is caused in the number of fused points in thickness direction of the nonwoven fabric 10. Accordingly, the front surface of the nonwoven fabric becomes easily movable in the direction along the front surface. The fiber amount toward the outer surface fiber layer 2 on the second surface side Z2 shaped in the top portion of the projection 121 of the support male material 120 becomes smaller than toward the outer surface fiber layer 1 on the first surface side Z1 shaped in the bottom portion of the concave portion 122 of the support male material 120. Therefore, the front surface of the nonwoven fabric becomes easily movable in the direction along the front surface.

With regard to the above embodiments, the present invention further discloses nonwoven fabrics and absorbent articles described below.

<1>

A nonwoven fabric, comprising a movable layer having front and back surfaces of the nonwoven fabric,

wherein the movable layer has a movable zone, in which one surface of the front and back surfaces is movable by 5 mm or more in a direction along the one surface, relative to the other surface.

<2>

The nonwoven fabric according to the above item <1>, wherein a moving range of the movable layer in a direction along the surface is 5 mm or more and 10 mm or less, preferably 6 mm or more, and more preferably 7 mm or more; and preferably 9 mm or less, and more preferably 8 mm or less.

<3>

The nonwoven fabric according to the above item <1> or <2>, wherein a moving range of the movable layer is measured based on the following [Method for measuring moving range of surface of nonwoven fabric]:

### [Method for Measuring Moving Range of Surface of Nonwoven Fabric]

(i) Preparation of measuring sample:

as a measuring sample, a nonwoven fabric sample having a size of 50 mm×50 mm is arranged; an adhesive is applied onto a whole surface of a mounting paper, which is called as a mount on the back surface side of the nonwoven fabric sample, to form an adhesive layer, and the back surface of the nonwoven fabric sample is adhered onto the adhesive layer and fixed thereonto; and an adhesive is applied onto a whole surface of another mounting paper, which is called as a mount on the front surface side of the nonwoven fabric sample, to form an adhesive layer, and the front surface of the nonwoven fabric sample is adhered onto the adhesive layer and fixed thereonto;

(ii) Measurement of moving range:

next, the mount on the back surface side is fixed on a base for measurement by using a fixing device; one end of a string for applying tensile force to the front surface of the nonwoven fabric sample in one direction along the front surface is attached to the mount on the front surface side; the other end of the string is vertically dangled downward through a rotatable pulley; during measurement, 50 g of weight is attached to the other end of the string so as to be suspended; accordingly, when the weight is attached to the other end of the string, the string pulls the mount on the front surface side in a direction along the front surface of the nonwoven fabric sample by gravity of the weight;

with regard to the measurement, first, an initial position of the nonwoven fabric sample is measured in a state without attaching the weight to obtain a measured value M1; then, the weight is attached thereto, and the weight is gently released; thus, the front surface of the nonwoven fabric sample is pulled in the direction along the front surface (pulley direction) by the weight;

the weight is released and the movement of the front surface of the nonwoven fabric sample is stopped, and then a stopping position of the nonwoven fabric sample is measured to obtain a measured value M2; and then, a moved amount of the front surface of the nonwoven fabric sample is obtained by calculating a difference between the measured value M2 and the measured value M1, and this amount is taken as the moving range of the front surface of the nonwoven fabric.

<4>

The nonwoven fabric according to any one of the above items <1> to <3>, wherein the number of fused points between the constituent fibers in the movable layer is lower in a region on an internal side of the movable layer than in the regions on the front surface side or the back surface side of the movable layer.

<5>

The nonwoven fabric according to any one of the above items <1> to <4>, wherein the region on the internal side of the movable layer means a region interposed between the front surface side and the back surface side of the movable layer.

<6>

The nonwoven fabric according to any one of the above items <1> to <5>, comprising a convex portion projecting from a reference surface of the nonwoven fabric in thickness direction,

wherein an external angle of a wall portion of the convex portion relative to the reference surface is 110° or less.

<7>

The nonwoven fabric according to the above item <6>, wherein the reference surface is a plane when the nonwoven fabric is extended and placed on the plane.

<8>

The nonwoven fabric according to the above item <6> or <7>, wherein the external angle of the wall portion forming the convex portion has external angles θ1 and θ2,

wherein the external angle θ1 is the angle between the reference surface and a straight line passing through an upper end portion and a lower end portion of the wall portion in a vertical cross section in the center of the concave portion of the concavo-convex portion along one direction of the nonwoven fabric,

and the external angle θ2 is the angle between the reference surface and a the straight line passing through an upper end portion and a lower end portion of the wall portion in a vertical cross section, being perpendicular to the above vertical cross section, in the center of the concave portion of the concavo-convex portion along a direction perpendicular to the one direction; and

wherein both external angles θ1 and θ2 are 110° or less.

<9>

The nonwoven fabric according to any one of the above items <6> to <8>, wherein the external angle is 60° or more and 110° or less, preferably 70° or more, and more preferably 80° or less; and preferably 100° or less, and more preferably 90° or less.

<10>

The nonwoven fabric according to the above item <8> or <9>, wherein the external angle θ1 measured from the one direction of the wall portion is in the comparable level relative to the external angle θ2 measured from the direction perpendicular to the one direction.

<11>

The nonwoven fabric according to the above item <10>, wherein the comparable level between the external angle θ1 and the external angle θ2 means that a difference between both angles is 0° or more and 10° or less, preferably 8° or less, more preferably 6° or less, and further preferably 4° or less.

<12>

The nonwoven fabric according to the above item <10>, wherein the comparable level between the external angle θ1 and the external angle θ2 means that a difference between both angles is 0° or more and 4° or less.

<13>

The nonwoven fabric according to any one of the above items <1> to <12>, formed of one sheet of nonwoven fabric.

<14>

The nonwoven fabric according to the above item <13>, wherein the one sheet of nonwoven fabric has no fibers melted in a film form.

<15>

The nonwoven fabric according to any one of the above items <1> to <14>, wherein the number of constituent fibers per unit area in a region on an internal side of the movable layer is 40% or more and 80% or less of the number of constituent fibers per unit area in the region on the front surface side or the back surface side of the movable layer.

<16>

The nonwoven fabric according to any one of the above items <1> to <15>, wherein the number of constituent fibers per unit area in a region of an internal side of the movable layer is 40% or more and 80% or less, preferably 45% or more, and more preferably 50% or more; and preferably 75% or less, and more preferably 70% or less, of the number of constituent fibers per unit area in the region on the front surface side or the back surface side of the movable layer.

<17>

The nonwoven fabric according to the above item <16>, wherein the number of constituent fibers per unit area in the region on the internal side of the movable layer is 40% or more and 80% or less, preferably 45% or more, and more preferably 50% or more; and preferably 75% or less, and more preferably 70% or less, of the number of constituent fibers per unit area in each of the region on the front surface side and the back surface side of the movable layer.

<18>

The nonwoven fabric according to any one of the above items <1> to <17>, wherein the number of fused points between the constituent fibers per unit area in a region on an internal side of the movable layer is 30% or more and 70% or less of the number of fused points between the constituent fibers per unit area in the region on the front surface side or the back surface side of the movable layer.

<19>

The nonwoven fabric according to any one of the above items <1> to <18>, wherein the number of fused points between the constituent fibers per unit area in a region on an internal side of the movable layer is 30% or more and 70% or less, preferably 35% or more, and more preferably 40% or more; and preferably 65% or less, and more preferably 60% or less of the number of fused points between the constituent fibers per unit area in the region on the front surface side or the back surface side of the movable layer.

<20>

The nonwoven fabric according to the above item <19>, wherein the number of fused points between the constituent fibers per unit area in the region on the internal side of the movable layer is 30% or more and 70% or less, preferably 35% or more, and more preferably 40% or more; and preferably 65% or less, and more preferably 60% or less of the number of fused points between the constituent fibers per unit area in each of the region on the front surface side and the back surface side of the movable layer.

<21>

The nonwoven fabric according to any one of the above items <1> to <20>, wherein a fiber orientation parameter in a region on an internal side of the movable layer is 1.1 times or more and 1.4 times or less to the fiber orientation parameter in the region on the front surface side or the back surface side of the movable layer.

<22>

The nonwoven fabric according to any one of the above items <1> to <21>, wherein a fiber orientation parameter in a region on an internal side of the movable layer is 1.1 times or more and 1.4 times or less, preferably 1.15 times or more, and more preferably 1.2 times or more; and preferably 1.35 times or less, and more preferably 1.3 times or less to the fiber orientation parameter in the region on the front surface side or the back surface side of the movable layer.

<23>

The nonwoven fabric according to the above item <22>, wherein a fiber orientation parameter in the region on the internal side of the movable layer is 1.1 times or more and 1.4 times or less, preferably 1.15 times or more, and more preferably 1.2 times or more; and preferably 1.35 times or less, and more preferably 1.3 times or less to the fiber orientation parameter in the region on each of the front surface side and the back surface side of the movable layer.

<24>

The nonwoven fabric according to any one of the above items <1> to <23>, comprising a difference in a basis weight amount in the nonwoven fabric,

wherein the nonwoven fabric has, in a region on an internal side of the movable layer, a region in which the basis weight amount is smaller than the amount of the region on the front surface side or the back surface side of the movable layer.

<25>

The nonwoven fabric according to any one of the above items <1> to <24>, comprising constituent fibers having a sheath-core structure, and a difference in a sheath-core ratio of the fibers in the sheath-core structure in the nonwoven fabric,

wherein the nonwoven fabric has, in a region on an internal side of the movable layer, a region in which a sheath ratio is smaller than the sheath ratio in the region on the front surface side or the back surface side of the movable layer.

<26>

The nonwoven fabric according to any one of the above items <1> to <25>, comprising a difference in the number of crimped fibers per unit area in the nonwoven fabric,

wherein the nonwoven fabric has, in a region on an internal side of the movable layer, a region in which the number of crimped fibers is smaller than the number of crimped fibers in the region on the front surface side or the back surface side of the movable layer.

<27>

The nonwoven fabric according to any one of the above items <1> to <26>, comprising a difference in a fiber diameter in the nonwoven fabric,

wherein the nonwoven fabric has, in a region on an internal side of the movable layer, a region in which the fiber diameter is larger than the diameter in the region on the front surface side or the back surface side of the movable layer.

<28>

The nonwoven fabric according to any one of the above items <1> to <27>, comprising a difference in a ratio of thermal expansion and contraction of the constituent fibers in the nonwoven fabric,

wherein the nonwoven fabric has, in a region on an internal side of the movable layer, a region in which the fibers are further thermally stretchable than a level in the region on the front surface side or the back surface side of the movable layer.

<29>

An absorbent article comprising the nonwoven fabric according to any one of the above items <1> to <28>.

<30>

An absorbent article, wherein the nonwoven fabric according to any one of the above items <1> to <28> is used for the topsheet.

### EXAMPLES

Hereinafter, the present invention will be described more in detail with reference to Examples, but the present invention is not limited thereto. Further, a term “%” in the Examples is based on mass unless otherwise noted.

### Example 1

A nonwoven fabric shown in FIG. 3 was prepared by using thermoplastic fibers of a sheath-core type (polyethylene terephthalate (PET) (core):polyethylene (PE) (sheath)=5:5 (mass ratio)) and having a fineness of 1.2 dtex according to an air-through production method including a production step shown in FIG. 12. The resultant material was taken as a nonwoven fabric sample in Example 1. Blowing treatment by first hot air W1 was applied thereto under conditions of a temperature of 160° C., an air speed of 54 m/s and a blowing time of 6 s. Blowing treatment by second hot air was applied thereto under conditions of a temperature of 160° C., an air speed of 6 m/s and a blowing time of 6 s.

### Example 2

A nonwoven fabric sample in Example 2 was prepared according to the same producing method as in Example 1 except that the basis weight amount was changed to the value shown in Table 1.

### Example 3

A nonwoven fabric sample in Example 3 was prepared according to the same producing method as in Example 1 except that the fineness was changed to the value shown in Table 1.

### Example 4

A nonwoven fabric sample in Example 4 was prepared according to the same producing method as in Example 1 except that thermoplastic fibers of a sheath-core type (polyethylene terephthalate (PET) (core):polyethylene (PE) (sheath)=7:3 (mass ratio)) and having a fineness of 3.2 dtex were used.

### Examples 5 to 7

A nonwoven fabric samples in Examples 5 to 7 were prepared according to the same producing method as in Examples 1 to 3 except that, with respect to the support female material 130 shown in FIG. 12, not a pattern in which the projections 131 were arranged to be lattice shape, but a pattern in which a plurality of the projections 131 was linked in a rotation axis direction of a ring-shaped disk at equal intervals to be drum shape, was arranged.

### Comparative Example 1

A nonwoven fabric produced by the method for producing a nonwoven fabric described in JP-A-2012-136791 was taken as a nonwoven fabric sample in Comparative Example 1.

### Comparative Example 2

A flat nonwoven fabric having constant thickness was prepared according to an air-through production method, and the resultant material was taken as a nonwoven fabric sample in Comparative Example 2.

### Comparative Example 3

A corrugated nonwoven fabric produced by tooth-space stretching processing according to the method for producing the nonwoven fabric in the invention described in JP-A-2016-79529 was taken as a nonwoven fabric sample in Comparative Example 3.

With respect to the above-described Examples and Comparative Examples, the “movable amount” was measured based on [Method for measuring moving range of front surface 10SA of nonwoven fabric 10] described above, and the “external angle of wall portion” was measured based on [Method for measuring external angle θ] described above. Further, with respect to the above-described Examples and Comparative Examples, each value was measured based on [Method for measuring fused point number], [Method for measuring the number of fibers], [Method for measuring fiber orientation parameter] and [Method for measuring apparent thickness of nonwoven fabric], and “basis weight amount of convex portion top portion” was measured based on [Method for measuring basis weight amount of nonwoven fabric].

Furthermore, with respect to the above-described Examples, a test of “recoverability after one day compression” was conducted as described later.

That is, a nonwoven fabric was sandwiched between two acrylic plates together with a washer having thickness of 0.7 mm, a weight (20 kg) was placed thereon, and a load was applied to compress the nonwoven fabric to thickness of 0.7 mm. After standing for one day in this state, the weight and the acrylic plates were removed from the nonwoven fabric, and after 10 minutes the apparent thickness of the nonwoven fabric was measured. From this measurement value and an apparent thickness of the nonwoven fabric before compression previously measured, recovery rate of the thickness of the nonwoven fabric was calculated to evaluate the recoverability after one day compression of the nonwoven fabric.

TABLE 1

| Item | | | Example 1 | Example 2 | Example 3 | Example 4 | Example 5 |
|---|---|---|---|---|---|---|---|
| Apparent thickness | | [mm] | 6.0 | 7.0 | 6.0 | 8.4 | 7.8 |
| Basis weight amount of convex top portion | | [g/m²] | 30 | 40 | 30 | 30 | 30 |
| Fineness of convex top portion | | [dtex] | 1.2 | 1.2 | 3.3 | 3.3 | 1.2 |
| Movable amount | MD | [mm] | 7 | 8 | 8 | 6 | 2 |
| | CD | [mm] | 7 | 6 | 6 | 6 | 6 |
| External angle | MD | [degree] | 95 | 95 | 95 | 95 | 100 |
| of wall portion | CD | [degree] | 95 | 95 | 95 | 95 | 0 |
| Formed of one sheet of nonwoven fabric | | | Y | Y | Y | Y | Y |
| The number | Front surface side | [number/mm²] | 171 | 133 | 89 | 94 | 150 |
| of fibers | Internal side | [number/mm²] | 89 | 97 | 51 | 38 | 99 |
| | Back surface side | [number/mm²] | 156 | 122 | 71 | 82 | 143 |
| | Internal side/ | [%] | 52 | 73 | 57 | 41 | 66 |
| | Front surface side | | | | | | |
| | Internal side/ | [%] | 57 | 79 | 71 | 47 | 70 |
| | Back surface side | | | | | | |
| The number | Front surface side | [piece/mm²] | 204 | 204 | 117 | 255 | 316 |
| of fused points | Internal side | [piece/mm²] | 117 | 128 | 71 | 77 | 209 |
| | Back surface side | [piece/mm²] | 255 | 189 | 117 | 138 | 383 |
| | Internal side/ | [%] | 58 | 63 | 61 | 30 | 66 |
| | Front surface side | | | | | | |
| | Internal side/ | [%] | 46 | 68 | 61 | 56 | 55 |
| | Back surface side | | | | | | |
| Fiber | Front surface side | [degree] | 56 | 55 | 55 | 58 | 53 |
| orientation | Internal side | [degree] | 64 | 62 | 66 | 81 | 60 |
| parameter | Back surface side | [degree] | 56 | 51 | 60 | 63 | 54 |
| | Internal side/ | [times] | 1.14 | 1.13 | 1.20 | 1.40 | 1.13 |
| | Front surface side | | | | | | |
| | Internal side/ | [times] | 1.14 | 1.22 | 1.10 | 1.29 | 1.11 |
| | Back surface side | | | | | | |

| | | | | | | |
|---|---|---|---|---|---|---|
| Recoverability after one day compression | [%] | 52 | 65 | 63 | 81 | 66 |

| | | | | | Comparative | Comparative | Comparative |
|---|---|---|---|---|---|---|---|
| Item | | | Example 6 | Example 7 | Example 1 | Example 2 | Example 3 |
| Apparent thickness | | [mm] | 8.1 | 7.2 | 3.0 | 3.0 | 2.5 |
| Basis weight amount of convex top portion | | [g/m²] | 40 | 30 | 30 | 30 | 70 |
| Fineness of convex top portion | | [dtex] | 1.2 | 3.3 | 1.2 | 1.2 | 3.3 |
| Movable amount | MD | [mm] | 1 | 2 | 2 | 1 | 0.5 |
| | CD | [mm] | 6 | 5 | 2 | 1 | 2 |
| External angle | M | [degree] | 100 | 100 | 110 | 0 | 110 |
| of wall portion | CD | [degree] | 0 | 0 | 110 | 0 | 0 |
| Formed of one sheet of nonwoven fabric | | | Y | Y | Y | Y | N |
| The number | Front surface side | [number/mm²] | 122 | 84 | 138 | 186 | 135 |
| of fibers | Internal side | [number/mm²] | 74 | 66 | 179 | 179 | 120 |
| | Back surface side | [number/mm²] | 135 | 97 | 224 | 184 | 105 |
| | Internal side/ | [%] | 60 | 79 | 130 | 96 | 89 |
| | Front surface side | | | | | | |
| | Internal side/ | [%] | 55 | 68 | 80 | 97 | 115 |
| | Back surface side | | | | | | |
| The number | Front surface side | [piece/mm²] | 321 | 224 | 224 | 209 | 153 |
| of fused points | Internal side | [piece/mm²] | 189 | 128 | 255 | 179 | 128 |
| | Back surface side | [piece/mm²] | 393 | 385 | 398 | 475 | 122 |
| | Internal side/ | [%] | 59 | 57 | 114 | 85 | 83 |
| | Front surface side | | | | | | |
| | Internal side/ | [%] | 48 | 33 | 64 | 38 | 104 |
| | Back surface side | | | | | | |
| Fiber | Front surface side | [degree] | 54 | 54 | 57 | 53 | 51 |
| orientation | Internal side | [degree] | 62 | 70 | 57 | 50 | 53 |
| parameter | Back surface side | [degree] | 55 | 60 | 53 | 53 | 52 |
| | Internal side/ | [times] | 1.15 | 1.30 | 1.00 | 0.94 | 1.04 |
| | Front surface side | | | | | | |
| | Internal side/ | [times] | 1.13 | 1.17 | 1.08 | 0.94 | 1.02 |
| | Back surface side | | | | | | |

| | | | |
|---|---|---|---|
| Recoverability after one day compression | [%] | 71 | 71 |

In Table 1, “Y” shows that the sample is formed of one sheet of nonwoven fabric, and “N” shows a material formed by bonding nonwoven fabrics.

The following results were obtained from Table 1. The nonwoven fabrics in Examples 1 to 7 had the movable amount of 5 mm or more, which was significantly longer than the amount in any of the nonwoven fabrics in Comparative Examples 1 to 3. Therefore, the nonwoven fabrics in Examples 1 to 7 had excellent followability to movement of a skin surface. Moreover, by the followability, it was found that the nonwoven fabrics in Examples 1 to 7 may suppress chafing of the nonwoven fabric onto the skin surface as caused by the movement of the skin surface. Moreover, the nonwoven fabrics in Examples 1 to 7 were smaller, in both the number of fibers and the number of fused points, in a center of thickness (internal side of the movable layer) than on the front surface side or the back surface side of the movable layer, in comparison with the nonwoven fabrics in Comparative Examples 1 to 3. Therefore, in the nonwoven fabrics in Examples 1 to 7, the fibers on the internal side of the movable layer became further easily movable, and the movable layer became further easily movable than in the nonwoven fabrics in Comparative Examples 1 to 3. Further, the nonwoven fabrics in Examples 1 to 7 had higher fiber orientation parameter in the center of thickness (internal side of the movable layer) than on the front surface side or the back surface side in comparison with the nonwoven fabrics in Comparative Examples 1 to 3, and therefore the front surface side or the back surface side was further easily movable. Therefore, a moving range in the movable layer in the nonwoven fabrics in Examples 1 to 7 was further widened than the moving range in the nonwoven fabrics in Comparative Examples 1 to 3, and therefore the above-described effect on the skin surface was further produced.

Furthermore, among Examples 1 to 7, Example 4 using the sheath-core type conjugate fibers in which a mass ratio of PE (temperature of glass transition component is lower in comparison with PET being core resin) being sheath resin was decreased was excellent in recoverability after one day compression, and it was found that the nonwoven fabric in Example 4 have high recoverability of the thickness even after the nonwoven fabric is compressed with a packing and the like.

Having described our invention as related to this embodiments and Examples, it is our intention that the invention not be limited by any of the details of the description, unless otherwise specified, but rather be construed broadly within its spirit and scope as set out in the accompanying claims.

This application claims priority on Patent Application No. 2017-168003 filed in Japan on Aug. 31, 2017, which is entirely herein incorporated by reference.

### DESCRIPTION OF SYMBOLS

1 Outer surface fiber layer on first surface side
2 Outer surface fiber layer on second surface side
3 Wall portion
4 Movable layer
4S Front surface side
4B Back surface side
4M Internal side of movable layer
10 Nonwoven fabric
10SA Front surface
10SB Back surface
10SS Reference surface
Z1 First surface side
Z2 Second surface side
SK Skin surface
θ, θ1, θ2 External angle

1. A nonwoven fabric, comprising a movable layer having front and back surfaces of the nonwoven fabric,
wherein the movable layer has a movable zone, in which one surface of the front and back surfaces is movable by 5 mm or more in a direction along the one surface, relative to the other surface.

2. The nonwoven fabric according to claim 1, wherein a moving range of the movable layer in a direction along the surface is 5 mm or more and 10 mm or less, preferably 6 mm or more, and more preferably 7 mm or more; and preferably 9 mm or less, and more preferably 8 mm or less.

3. The nonwoven fabric according to claim 1, wherein the number of fused points between the constituent fibers in the movable layer is lower in a region on an internal side of the movable layer than in the regions on the front surface side or the back surface side of the movable layer.

4. The nonwoven fabric according to claim 1, comprising a convex portion projecting from a reference surface of the nonwoven fabric in thickness direction,
wherein an external angle of a wall portion of the convex portion relative the reference surface is 110° or less.

5. The nonwoven fabric according to claim 4,
wherein the external angle of the wall portion forming the convex portion has external angles θ1 and θ2,
wherein the external angle θ1 is the angle between the reference surface and a straight line passing through an upper end portion and a lower end portion of the wall portion in a vertical cross section in the center of the concave portion of the concavo-convex portion along one direction of the nonwoven fabric, and the external angle θ2 is the angle between the reference surface and a straight line passing through an upper end portion and a lower end portion of the wall portion in a vertical cross section, being perpendicular to the above vertical cross section, in the center of the concave portion of the concavo-convex portion along a direction perpendicular to the one direction; and
wherein both external angles θ1 and θ2 are 110° or less.

6. The nonwoven fabric according to claim 5, wherein the external angle θ1 measured from the one direction of the wall portion is in the comparable level relative to the external angle θ2 measured from the direction perpendicular to the one direction.

7. The nonwoven fabric according to claim 1, formed of one sheet of nonwoven fabric.

8. The nonwoven fabric according to claim 1, wherein the number of constituent fibers per unit area in a region on an internal side of the movable layer is 40% or more and 80% or less of the number of constituent fibers per unit area in the region on the front surface side or the back surface side of the movable layer.

9. The nonwoven fabric according to claim 1, wherein the number of constituent fibers per unit area in the region on the internal side of the movable layer is 40% or more and 80% or less, preferably 45% or more, and more preferably 50% or more; and preferably 75% or less, and more preferably 70% or less, of the number of constituent fibers per unit area in each of the region on the front surface side and the back surface side of the movable layer.

10. The nonwoven fabric according to claim 1, wherein the number of fused points between the constituent fibers per unit area in a region on an internal side of the movable layer is 30% or more and 70% or less of the number of fused points between the constituent fibers per unit area in the region on the front surface side or the back surface side of the movable layer.

11. The nonwoven fabric according to claim 1, wherein the number of fused points between the constituent fibers per unit area in the region on the internal side of the movable layer is 30% or more and 70% or less, preferably 35% or more, and more preferably 40% or more; and preferably 65% or less, and more preferably 60% or less of the number of fused points between the constituent fibers per unit area in each of the region on the front surface side and the back surface side of the movable layer.

12. The nonwoven fabric according to claim 1, wherein a fiber orientation parameter in a region on an internal side of the movable layer is 1.1 times or more and 1.4 times or less to the fiber orientation parameter in the region on the front surface side or the back surface side of the movable layer.

13. The nonwoven fabric according to claim 1, wherein a fiber orientation parameter in the region on the internal side of the movable layer is 1.1 times or more and 1.4 times or less, preferably 1.15 times or more, and more preferably 1.2 times or more; and preferably 1.35 times or less, and more preferably 1.3 times or less to the fiber orientation parameter in the region on each of the front surface side and the back surface side of the movable layer.

14. The nonwoven fabric according to claim 1, comprising a difference in a basis weight amount in the nonwoven fabric,
wherein the nonwoven fabric has, in a region on an internal side of the movable layer, a region in which the basis weight amount is smaller than the amount of the region on the front surface side or the back surface side of the movable layer.

15. The nonwoven fabric according to claim 1, comprising constituent fibers having a sheath-core structure, and a difference in a sheath-core ratio of the fibers in the sheath-core structure in the nonwoven fabric,
wherein the nonwoven fabric has, in a region on an internal side of the movable layer, a region in which a sheath ratio is smaller than the sheath ratio in the region on the front surface side or the back surface side of the movable layer.

16. The nonwoven fabric according to claim 1, comprising a difference in the number of crimped fibers per unit area in the nonwoven fabric,
wherein the nonwoven fabric has, in a region on an internal side of the movable layer, a region in which the number of crimped fibers is smaller than the number of crimped fibers in the region on the front surface side or the back surface side of the movable layer.

17. The nonwoven fabric according to claim 1, comprising a difference in a fiber diameter in the nonwoven fabric,
wherein the nonwoven fabric has, in a region on an internal side of the movable layer, a region in which the fiber diameter is larger than the diameter in the region on the front surface side or the back surface side of the movable layer.

18. The nonwoven fabric according to claim 1, comprising a difference in a ratio of thermal expansion and contraction of the constituent fibers in the nonwoven fabric,
wherein the nonwoven fabric has, in a region on an internal side of the movable layer, a region in which the fibers are further thermally stretchable than a level in the region on the front surface side or the back surface side of the movable layer.

19. An absorbent article comprising the nonwoven fabric according to claim 1.

20. An absorbent article, wherein the nonwoven fabric according to claim 1 is used for the topsheet.

21-30. (canceled)
